(19) United States
(12) Patent Application Publication
(10) Pub. No.: US 2026/0305652 A1
(43) Pub. Date: Oct. 8, 2026

(54) Culturing Methylotrophic Microorganisms to Produce a Biomass

(71) Applicant: Farmless Holding B.V., Amsterdam (NL)

(72) Inventors: Adnan Oner, Amsterdam (NL); Maria Claudia Cuellar Soares, Amsterdam (NL); Wanqing Jia, Amsterdam (NL); Ana Maria Zetty Arenas, Amsterdam (NL)

(21) Appl. No.: 19/482,105

(22) PCT Filed: May 9, 2024
(86) PCT No.: PCT/EP2024/062878

§ 371 Date: Nov. 6, 2025

(30) Foreign Application Priority Data
May 9, 2023 (GB) 2306850.5

(51) Int. Cl. A23J 3/20; A23J 1/00; A23J 3/22; C12N 1/02; C12N 1/16; C12N 1/20; C12P 21/00; C12R 1/01; C12R 1/645

(52) U.S. Cl. CPC A23J 3/20; A23J 1/008; A23J 3/227; C12N 1/02; C12N 1/16; C12N 1/20; C12P 21/00; C12R 2001/01; C12R 2001/645

(57) ABSTRACT

The invention provides methods of producing single cell protein products by culturing methylotrophic microorganisms The methods provide culturing conditions and processing methods that can be used to alter the properties of the single cell protein products. Also provided a single cell protein products produced by the methods and food products including the single cell protein products.

The present invention relates to methods of culturing methylotrophic microorganisms to produce a biomass. The invention also relates to products including the biomass and/or products derived from the biomass and uses thereof.

### BACKGROUND

The use of alternative protein sources such as plant and microorganism derived proteins is gaining more importance given the environmental impact of traditional farming, as well as the shift in consumer consciousness of how and where their food is cultivated.

Examples of alternative proteins include plant proteins such as pea and soy protein as well as fungal proteins such as mycoproteins. Even though production of these proteins have a lower impact on the environment, they still have a substantial carbon footprint. For example, the process of arable farming, used to produce agricultural derived proteins, is around 21-37% of CO₂ equivalent emissions, occupies half of all habitable land on the planet and is the leading cause of deforestation and biodiversity loss, causing over 90% of tropical deforestation. Agriculture is also vulnerable to production shocks due to warmer weather and more frequent climate extremes. In this process (plant-based food), only ~1-2% of incoming sunlight is turned into biomass and around 0.25-0.5% into edible plant based food. And when given to animals over 99.99% of the energy of sunlight is wasted. Therefore, there is a need for more efficient means to produce protein alternatives.

Imperial chemical industries developed an animal feed derived from methylotrophic bacteria referred to as “Pruteen®”. For example, methods of culturing protein from methylotrophic bacteria have been described in U.S. Pat. Nos. 3,989,594A, 4,306,026A, 4,317,843A, and 3,989,594A.

However, the processes and products described therein provide no suggestion of how they may be adapted to provide protein suitable for human consumption. Nor is there any suggestion of how to improve the efficiency and environmental impact of such methods.

There is also a need for alternative proteins for use in vegan or vegetarian food analogues, such as dairy, egg, seafood and meat analogues. There is currently no methylotrophic derived protein that has been optimised for use in such analogues.

There is a need for improved methods of producing single cell protein and products containing single cell protein.

There is a need for single cell protein biomass for food products with improved organoleptic and/or physical properties.

There is a need for more efficient and/or environmentally friendly production of consumable protein.

### BRIEF SUMMARY OF THE DISCLOSURE

The invention aims to provide methods of producing methylotrophic derived protein containing products that have improved properties. In particular improved properties for use as an analogue for animal derived proteins and other food products.

It is also an aim of the invention to provide methods of producing methylotrophic derived protein that has a reduced environmental impact.

The inventors have found that by using specific culturing conditions, culturing methods and/or post-culturing (i.e. downstream) processing methods can provide a methylotrophic microorganism biomass or protein product that has tuneable advantageous properties for use in meat analogues, dairy analogues and other food products.

The inventors have also found that the methods herein can have reduced environmental impact and/or improved efficiency by the use of e-methanol as a feedstock for culturing the methylotrophic microorganism.

The inventors have also found that by configuring culturing conditions and/or post-culture processing methods, a single strain of microorganism is capable of providing protein and protein containing products (e.g. biomass and culture broth) that have different properties allowing for their use in multiple alternative food products.

In one aspect of the invention there is provided a method of producing a single cell protein product for incorporation into a food product for human consumption, the single cell protein product comprising single cell protein and a concentration of Cytochrome C of at least 10×10⁻³ mM, the method comprising;
culturing the methylotrophic microorganism in a culture medium comprising methanol at a dissolved oxygen concentration from about 15% to 50%;
wherein the culturing conditions are configured to induce the methylotrophic microorganism to produce protein and Cytochrome C within a biomass and/or the culture medium to produce a culture broth; and/or
to provide the single cell protein product with properties configured for use in the food product:
wherein the culture broth comprises the concentration of Cytochrome C of at least 10×10⁻³ mM and/or wherein the biomass comprises a concentration of Cytochrome C of at least 200 pmol/mg of dry material of biomass.

In one aspect of the invention there is provided a method of producing a culture broth or product derived therefrom comprising a concentration of Cytochrome C of at least 10×10⁻³ mM, the method comprising;
culturing a methylotrophic microorganism in a culture medium comprising methanol at a dissolved oxygen concentration from about 15% to 50%;
wherein the culturing conditions are configured to induce the methylotrophic microorganism to produce protein and Cytochrome C within a biomass and/or the culture medium to produce the culture broth.

In certain embodiments, the method further comprises harvesting the single cell protein product. In certain embodiments, harvesting comprises at least partially disrupting the cells of the methylotrophic microorganism in order to release the cellular components thereof. In certain embodiments, at least partially disrupting the cells comprises acidifying the cultured biomass and/or culture broth prior to separating the cultured biomass from the culture broth to provide an acidified culture broth comprising at least a portion of the Cytochrome C. In certain embodiments, at least partially disrupting the cells comprises homogenising the cultured biomass and/or culture broth. In certain embodiments, at least partially disrupting the cells comprises heating the cultured biomass and/or culture broth. In certain embodiments, at least partially disrupting the cells comprises enzymatic treatment of the cultured biomass and/or culture broth.

In certain embodiments, harvesting comprises centrifuging the culture broth at a force of more than 2000 g to provide a supernatant comprising at least a portion of the Cytochrome C and a pellet comprising the biomass. In certain embodiments, the biomass comprises a concentration of Cytochrome C of at least 200 pmol/mg of dry material of biomass.

In certain embodiments, the method further comprises processing:
the protein of the biomass; the protein of the culture broth; the biomass; and/or the culture broth.

In certain embodiments, the harvesting further comprises processing:
the protein of the biomass; the protein of the culture broth; the biomass; the culture broth; the acidified culture broth; and/or the supernatant.

In certain embodiments, processing comprises filtration of the supernatant to provide a concentrated retentate.

In certain embodiments, the method further comprises freezing the concentrated retentate.

In certain embodiments, the method further comprises drying the concentrated retentate. In certain embodiments, drying comprises one or more of freeze drying and/or spray drying and/or other drying methods.

In one aspect of the invention there is provided a method of producing a single cell protein product for incorporation into a food product for human consumption, the single cell protein product comprising single cell protein and a concentration of Cytochrome C of at least 10×10⁻³ mM, the method comprising;
culturing a methylotrophic microorganism in a culture medium comprising methanol at a dissolved oxygen concentration from about 15% to 50%;
wherein the culturing conditions are configured to induce the methylotrophic microorganism to produce protein and Cytochrome C within a biomass and/or the culture medium to produce a culture broth;
at least partially disrupting the cells of the cultured methylotrophic microorganism;
centrifuging the at least partially disrupted cells and culture media to produce a supernatant and pellet, the pellet comprising the biomass;
wherein the supernatant comprises a concentration of Cytochrome C of at least 10×10⁻³ mM and/or wherein the pellet comprises a concentration of Cytochrome C of at least 200 pmol/mg of dry material of biomass.

In one aspect of the invention there is provided a method of producing cytochrome C the method comprising;
culturing a methylotrophic microorganism in a culture medium comprising methanol at a dissolved oxygen concentration from about 15% to 50%;
wherein the culturing conditions are configured to induce the methylotrophic microorganism to produce protein and Cytochrome C within a biomass and/or the culture medium to produce a culture broth;
at least partially disrupting the cells of the cultured methylotrophic microorganism;
centrifuging the at least partially disrupted cells of the culture broth to produce a supernatant and pellet, the pellet comprising the biomass;
wherein the supernatant comprises a concentration of Cytochrome C of at least 10×10⁻³ mM and/or wherein the pellet comprises a concentration of Cytochrome C of at least 200 pmol/mg of dry material of biomass.

In one aspect of the invention there is provided a method of producing a single cell protein product for incorporation into a food product for human consumption, the single cell protein product comprising single cell protein, the method comprising;
culturing one or more methylotrophic microorganism in a culture medium comprising methanol under culturing conditions for fermentation of the methanol;
wherein the culturing conditions are configured to induce the methylotrophic microorganism to produce protein within a biomass and/or the culture medium to produce a culture broth; and/or to provide the single cell protein product with properties configured for use in the food product.

In some embodiments, the single cell protein product comprises the biomass, the culture broth and/or the protein of the biomass and/or culture broth.

In some embodiments, the culturing conditions comprise exposing the methylotrophic microorganism to one or more conditions during culturing selected from:
dissolved oxygen concentration; pH; temperature; methanol concentration; nitrogen concentration; phosphorus concentration; and/or magnesium concentration optionally

In some embodiments, the one or more conditions comprises or further comprises one or more of:
aeration rate; stirring rate; nitrogen feed rate; methanol feed rate; and/or nutrient feed rate.

In some embodiments:
the methanol concentration comprises 0-10 g/L;
the dissolved oxygen concentration comprises 5 to 50%; or 15 to 45%;
the pH comprises a pH from about 5 to 7;
the nitrogen concentration comprises 0-1.2 g/L;
the temperature comprises from 20 to 70° C.;
the phosphorus concentration comprises from 0 to 2.0 g/l; and/or
the magnesium concentration comprises from 0 to 80 mg/l.

In some embodiments, the culturing conditions comprise stress conditions.

In some embodiments, the stress conditions comprise nitrogen limitation and/or oxygen limitation.

In some embodiments, the methanol comprises sustainable methanol.

In some embodiments, the methanol comprises one or more of e-methanol, bio-methanol, blue methanol and/or grey methanol.

In some embodiments, culturing comprises restricted fermentation and/or unrestricted fermentation.

In some embodiments, the culturing comprises:
a batch fermentation process;
a fed-batch fermentation process;
a continuous fermentation process;
a feed-and-draw fermentation process;
a perfusion fermentation process;
a sequential batch process; and/or
a cell recycle fermentation process.

In some embodiments, the methylotrophic microorganism comprises one or more of at least one yeast, at least one bacteria and/or at least one archaea.

In some embodiments, the methylotrophic yeast is one or more selected from the group consisting of Acidomonas, Candida, Hansenula, Komagataella, Ogataea, Pichia, Rhodotorula, and/or Torulopsis.

In some embodiments, the methylotrophic bacterium is one or more selected from the group consisting of Acidomonas, Albibacter, Amycolatopsis, Ancylobacter, Arthrobacter, Bacillus, Chenggangzhangella, Clostridium, Hydrogenovibrio, Hyphomicrobium, Labrys, Leisinglera, Methanococcoides, Methanothermobacter, Methylacidiphilum, Methylibium, Methylobacillus, Methylobacterium, Methylorubrum, Methylocaldum, Methylocapsa, Methyloceanibacter, Methylocella, Methylococcus, Methylocystis, Methyloferula, Methylogaea, Methyloglobulus, Methylohalobius, Methylohalomonas, Methylomicrobium, Methylomonas, Methylonatrum, Methylophaga, Methylophilus, Methylopila, Methylorosula, Methylorubrum, Methylosarcina, Methylosinus, Methylosoma, Methylosphaera, Methylotenera, Methylothermus, Methyloversatilis, Methylovirgula, Methylovorus, Microvirga, Nocardia, Novimethylophilus, Oharaeibacter, Paracoccus, Pontibaca, Pseudomonas, Pyrinomonas, Rhodoblastus, Rhodococcus, Rubrivivax, Shinella, and/or Sporomusa.

In some embodiments, the methylotrophic archaea is one or more selected from Methanohalophilus, Methanolacinia, Methanolinea, Methanolobus, Methanomethylovorans, Methanosarcina, Methanobrevibacter, Methanothermococcus, Methanococcus, Methanotorris, Methanothermobacter Methanofervidicoccus, Methanosphaera, and/or Methanimicrococcus;

In some embodiments, the methylotrophic microorganism is a strain of Methylophilus methylotrophus.

In some embodiments, the method further comprises harvesting the cultured protein of the biomass and/or culture broth or harvesting the cultured biomass.

In some embodiments, harvesting comprises separating the protein from the cultured biomass and/or culture broth or separating the biomass from the culture broth.

In some embodiments, separating comprises one or more of flocculation, flotation, decanting, filtration and/or centrifugation.

In certain embodiments, the harvesting comprises at least partially disrupting the cells of the methylotrophic microorganism in order to release the cellular components thereof. In certain embodiments, at least partially disrupting the cells comprises acidifying the cultured biomass and/or culture broth prior to separating the cultured biomass from the culture broth to provide an acidified culture broth. In certain embodiments, at least partially disrupting the cells comprises homogenising the cultured biomass and/or culture broth. In certain embodiments, at least partially disrupting the cells comprises heating the cultured biomass and/or culture broth. In some embodiments, the harvesting comprises acidifying the cultured biomass and/or culture broth prior to separating the cultured biomass from the culture broth.

In some embodiments, acidification is done in the same vessel as the culturing or in a separate vessel as the culturing.

In some embodiments, acidification results in a pH in the range 3 to 6.

In some embodiments, the harvesting further comprises washing prior to separation and/or washing the separated biomass.

In some embodiments, the harvesting further comprises drying the separated protein of the biomass and/or culture broth or drying harvested the biomass.

In some embodiments, the methylotrophic microorganism is cultured using an unrestricted fermentation process and the single cell protein product has a viscosity of less than 200 mPa·s measured at 1S⁻¹.

In some embodiments, the methylotrophic microorganism is cultured using a restricted fermentation process and the single cell protein product has a viscosity of more than 300 mPa·s measured at 1S⁻¹.

In some embodiments, the method further comprises processing
the protein of the biomass; the protein of the culture broth; the biomass; and/or the culture broth.

In some embodiments, the harvesting further comprises processing:
the protein of the biomass; the protein of the culture broth; the biomass; and/or; the culture broth.

In some embodiments, the processing methods are configured to provide properties to the single cell protein product configured for use in the food product;

In some embodiments, the processing comprises one or more of:
centrifugation; homogenisation; acidification; drying; enzymatic treatment; cooling; heating; filtration; rehydrating; rotary evaporation; acid gelation; curdling; texturizing; and/or freezing.

In some embodiments, processing comprises;
centrifugation at force less than 10,0000 g, optionally wherein the protein of the biomass and/or culture broth or the biomass and/or culture broth (single cell protein product) has a viscosity of less than 200 mPa·s measured at 1S⁻¹ prior to centrifugation.

In some embodiments, processing comprises;
homogenisation and centrifugation at a force less than 10,0000 g, optionally wherein the protein of the biomass and/or culture broth or the biomass and/or culture broth (single cell protein product) has a viscosity of more than 300 mPa·s measured at 1S⁻¹ prior to homogenisation. In some embodiments, centrifugation is at a force of less than 6500 g. In some embodiments, centrifugation is at a force of less than 4000 g. In some embodiments, centrifugation is at a force of about 4000 g. In some embodiments, centrifugation is at a force of about 2000 g.

In some embodiments, processing comprises;
centrifugation at a force more than 10,000 g, optionally wherein the protein of the biomass and/or culture broth or the biomass and/or culture broth (single cell protein product) has a viscosity of more than 300 mPa·s measured at 1S⁻¹ prior to centrifugation. In some embodiments, centrifugation is at a force of more than 2000 g. In some embodiments, centrifugation is at a force of about 4000 g. In some embodiments, centrifugation is at a force of about 18,000 g. In some embodiments, centrifugation is at a force of about 20,000 g.

In some embodiments, the method further comprises drying the centrifuged protein of the biomass and/or culture broth or the biomass and/or culture broth.

In some embodiments, the method further comprises rehydrating the dried protein of the biomass and/or culture broth or the dried biomass and/or dried culture broth.

In some embodiments, supernatant from any of the centrifugations is acidified.

In some embodiments, the acidified supernatant is further centrifuged at a force less than 10,000 g.

In some embodiments, the properties are selected from one or more of: rheological properties, protein concentration, aesthetic properties, organoleptic properties, structural properties and/or physical properties.

In some embodiments, the food product is:
dairy analogue and the single cell protein product comprises a viscosity of less than 200 mPa·s measured at 1S⁻¹, a cream like texture and/or a white or off white colour.

In some embodiments, the food product is:
a meat analogue and the single cell protein product comprises a viscosity of more than 300 mPa·s measured at 1S⁻¹, an oil holding capacity (OHC) of more than 1.5 grams of oil per gram of single cell protein product, organoleptic properties mimetic of meat and/or appearance mimetic of meat.

In another aspect of the invention there is provided a single cell protein product produced by a method described herein.

In another aspect of the invention there is provided a single cell protein product comprising any of the properties described herein.

In some embodiments, the single cell protein product comprises a protein content determined by a Dumas method with a Nitrogen Conversion Factor of 4.88 of at least 45%.

In some embodiments:
the microorganism has been cultured using an unrestricted fermentation process and wherein the single cell protein product comprises:
viscoelastic properties;
a protein content of about 45%;
a water holding capacity of at least 2.5 gram water per gram single cell protein product;
an oil holding capacity of at least 1.0 gram oil per gram of single cell protein product
a creamy texture;
a white colour;
a viscosity of less than 200 mPa·s measured at 1S⁻¹ and/or solubility of at least 20%.

In some embodiments, the microorganism has been cultured using a restricted fermentation process and wherein the single cell protein product comprises:
viscoelastic properties;
a protein content of about 60%;
a water holding capacity of about 6 gram water per gram of single cell protein product;
an oil holding capacity of about 1.5 gram water per gram of single cell protein product;
a colour and/or appearance mimetic of animal meat;
a viscosity of more than 300 mPa·s measured at 1S⁻¹; and/or solubility of about 20%.

In another aspect there is provided a food product for human consumption comprising a single cell protein product as described herein.

In some embodiments, the food product comprises:
a dairy analogue; an egg analogue; a beverage; a soup; a sauce; a dairy product; a seafood analogue; and/or a meat analogue.

In some embodiments, the single cell protein product is produced by or from a microorganism has been cultured using an unrestricted fermentation process as described herein and the food product comprises a dairy analogue; an egg analogue; a beverage; a soup; a sauce; a seafood analogue; and/or a dairy product.

In some embodiments, the single cell protein product is produced by or from a microorganism has been cultured using a restricted fermentation process as described herein and the food product comprises a meat analogue.

In another aspect of the invention there is provided, single cell protein product for incorporation into a food product for human consumption, the single cell protein product comprising a concentration of Cytochrome C of at least 200 pmol/mg of dry material of single cell protein product; and wherein the single cell protein product comprises or is obtained from a biomass produced by culturing a methylotrophic microorganism.

In another aspect of the invention there is provided, a single cell protein product for incorporation into a food product for human consumption, the single cell protein product comprising a concentration of Cytochrome C of at least 10×10⁻³ mM; and wherein the single cell protein product comprises or is obtained from a culture broth produced by culturing a methylotrophic microorganism.

In some embodiments, the single cell protein product comprising the concentration of Cytochrome C is produced by a method described herein.

In some embodiments, the single cell protein product comprises or is obtained from a culture broth produced by culturing a methylotrophic microorganism

In some embodiments, the Cytochrome C is a product of culturing a methylotrophic microorganism. In some embodiments, the Cytochrome C has not been added from an exogenous source.

In some embodiments, the single cell protein product has been harvested and/or processed as described herein.

In some embodiments, the Cytochrome C is extracted from the culture broth and added into a single cell protein product after processing as described herein.

In some embodiments, the methylotrophic microorganism has been cultured using a fed-batch or continuous cultured method and a dissolved oxygen concentration from about 15% to 50%.

In some embodiments, the single cell protein product comprises or consists of the culture broth. In some embodiments, the single cell protein product is derived from the culture broth. In some embodiments, the culture broth comprises cultured methylotrophic microorganism and culture media. In some embodiments, the culture broth has been centrifuged and the supernatant is the single cell protein product. In some embodiments, the cells of the culture broth have been at least partially disrupted before centrifugation.

In some embodiments, the single cell protein product is the pellet produced by centrifugation of the culture broth and the pellet comprises a concentration of Cytochrome C of at least 200 pmol/mg of dry material of pellet. In some embodiments, the cells of the culture broth have been at least partially disrupted before centrifugation.

In another aspect of the invention there is provided a methylotrophic microorganism comprising a concentration of Cytochrome C of at least 200 pmol/mg of dry material of methylotrophic microorganism. In some embodiments, the microorganism produces or has produced a concentration of Cytochrome C of at least 200 pmol/mg of dry material of methylotrophic microorganism.

In some embodiments, the single cell protein product comprising the concentration of Cytochrome C or food product comprising the single cell protein product comprising the concentration of Cytochrome C comprises a pink, red, beige, off-white or reddish-brown colour.

In another aspect of the invention there is provided an use of sustainable methanol in a method of producing a composition comprising protein, a biomass or a single cell protein product cultured from a methylotrophic microorganism.

In some embodiments, the methanol is e-methanol.

In some embodiments, the method comprises a method as described herein.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Various aspects of the invention are described in further detail below.

### BRIEF DESCRIPTION OF THE FIGURES

Embodiments of the invention are further described hereinafter with reference to the accompanying drawings, in which:

FIG. 1 shows Rheological behaviour as determined by the viscoelastic modulus for pea protein isolate (PPII), pea protein concentrate (PPC), soy protein isolate (SPI), soy protein concentrate (SPC) and microbial biomass obtained from fed-batch fermentation as described in the Materials and Methods section.

FIG. 2 shows Rheological behaviour as determined by the viscoelastic modulus for microbial biomass cultivated under unrestricted (batch) and restricted (fed-batch) growth conditions, harvested by centrifugation, washed and freeze dried.

FIG. 3 shows Microbial protein appearance upon post-processing: a) after centrifugation and washing; b) after centrifugation and freeze drying; c) after centrifugation, washing and freeze drying.

FIG. 4 shows a flow through of post-culture processing. Pellet 1—Good solubility, suited to dairy/egg alternatives with applications for beverages, soup, salad dressings, ice-cream, whipped toppings etc; Pellets 2 and 3—Good Water/oil holding capacity, rheological properties suited to meat alternatives, soap/salad dressing etc; Pellet 4—Enhanced rheological properties, suited to meat alternatives, soap/salad dressing etc.

FIG. 5 shows downstream processing methods (post-culture processing), principles thereof and applications that the processed biomass may have.

FIG. 6 shows downstream processing methods (post-culture processing), principles thereof and applications that the processed biomass may have.

FIG. 7 shows relative increase in feeding rate and OD (logarithmic scale) for Tank 1 and Tank 4, both operated under oxygen limitation (DO=5%). Tank 4 displays restricted growth by an overfeeding strategy.

FIG. 8 shows relative increase in OD for Tank 1 and Tank 5, both operated at the same feeding rate. Tank 1 displays restricted growth by oxygen limitation (DO=5%) relative to Tank 5, operating under oxygen excess (DO=50%).

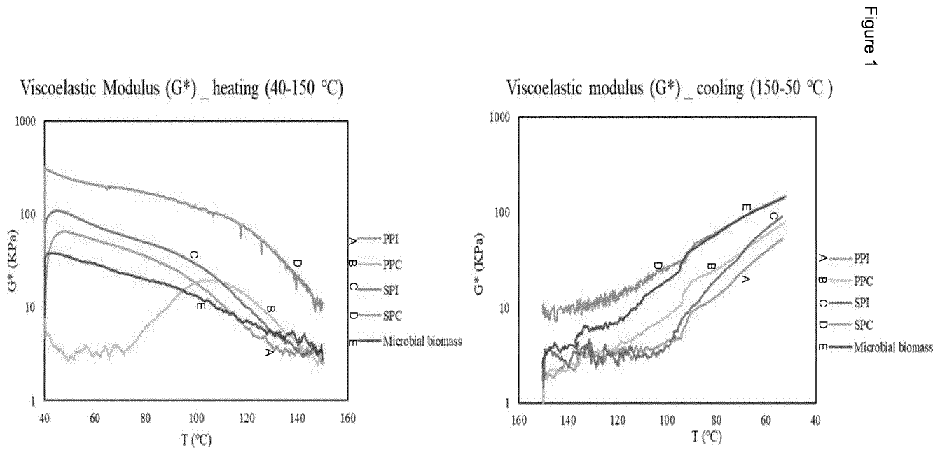

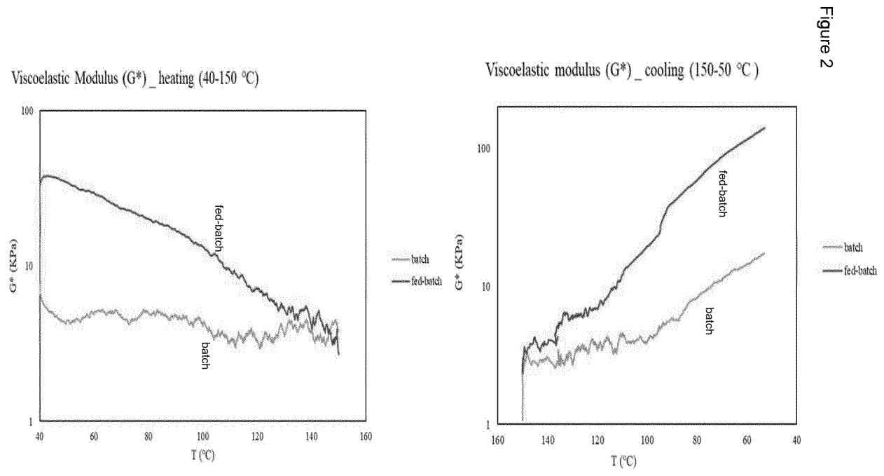

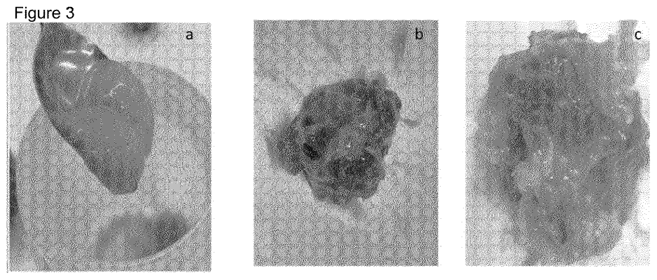

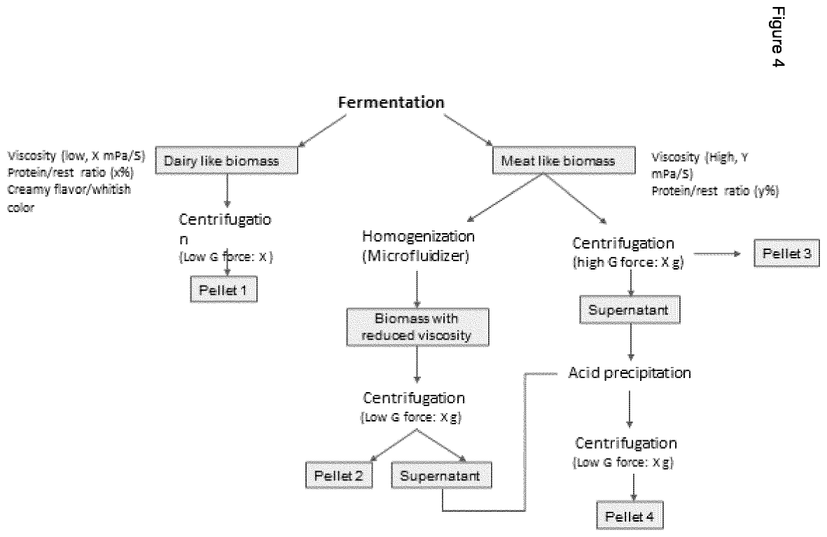

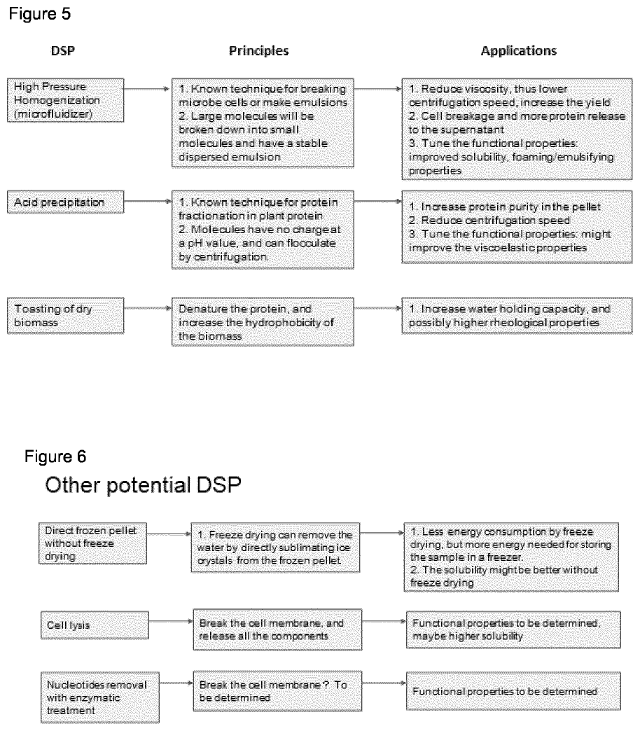

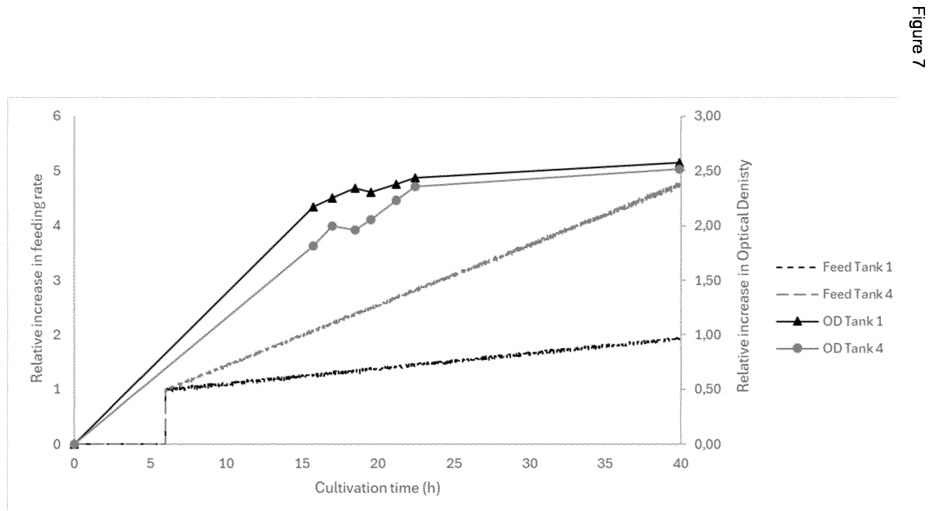

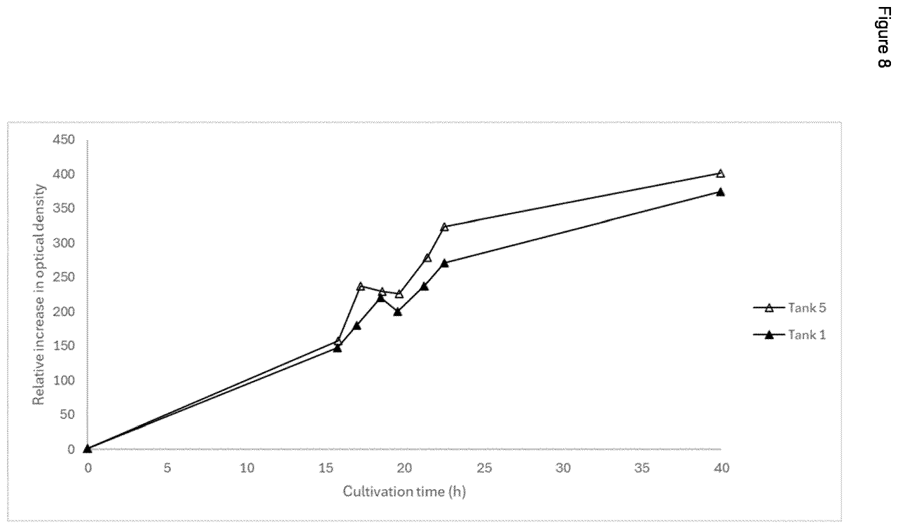

The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

Various aspects of the invention are described in further detail below.

### DETAILED DESCRIPTION

### Single Cell Protein Product

The methods provided herein provide products (i.e. compositions) that include protein derived from a single cell organism. As such, any products produced by the methods described herein, including produced by any one or more of culturing, harvesting and/or post-culture processing methods may be referred to as single cell protein products. The term “single cell protein product” is used to refer to any products of the methods described herein that include protein produced by the microorganism. The single cell protein products may include other components such as carbohydrates, lipids and other component parts of the microorganism. These other components may also impart properties to the single cell protein products that are of use in certain food products. For example, organoleptic properties (such a mouthfeel, smell, or taste), physical properties (such as texture, viscosity etc.) and/or aesthetic properties such as appearance and colour.

In some examples, the single cell protein product is a biomass produced by culturing the microorganism. The biomass may be processed to provide a protein concentrate or isolated protein. The biomass described herein and protein products derived therefrom may be referred to as “single cell protein products”. As used herein, the term “single-cell protein” refers to a crude or refined protein originating from a unicellular organism. In some cases, the term biomass is used to refer to a whole cell biomass that includes the single cell protein. In some examples, the single cell protein product is the protein harvested, isolated or purified from the biomass.

Methods of preparing a protein concentrate are described herein (e.g. acid precipitation) as well as being well known in the art. For example, see EP0252900A2 which is incorporated herein by reference.

Proteins may be isolated or purified from the biomass by any suitable methods. For example, by acid precipitation, density gradient centrifugation, dialysis, column chromatography, gel filtration, size exclusion, ion exchange, electrophoresis methods, and antibody based methods.

In some examples, only the protein element of the biomass may be recovered (i.e. harvested or processed). In some examples, protein is additionally and/or alternatively recovered from the culture broth. For example using any suitable method for recovery, isolation and/or purification of proteins known in the field. Such as, by acidification methods.

Therefore, in some examples, the single cell protein product is the culture broth. In some examples, the single cell protein product is the protein harvested, isolated or purified from the culture broth.

In some examples, the culture broth refers to the combination of cultured cells and culture media after culturing. As such, products derived from the culture broth may include cultured cells or parts thereof. In some examples, the harvesting or processing described herein may lead to disruption of the cultured cells. Therefore, culture media including cultured and disrupted cells may also be also referred to as culture broth. As such, the single cell protein product may be products obtained from processing the culture broth. For example, the supernatant or pellet produced by centrifugation of the culture broth before or after disruption of the cultured cells.

### Microorganisms

The present invention relates to a method of culturing methylotrophic microorganisms.

“Methylotroph” refers to microorganisms that can use reduced one-carbon compounds, such as but not limited to methanol, as a carbon source and/or as an electron donor for their growth. Examples of methylotrophic microorganisms include yeast, bacteria and archae. For example, those microorganisms derived from the genera: Methylosinus; Methylocystis; Methylomonas; Methylobacter; Methylococcus; and Methylobacterium. For example the methylotphic microgansim may be derived from Methanomonas; Pseudomonasl; Bacterium; Hyphomicrobium; Achromabacter; Protaminobacter; Vibrio; Rhodopseudomonas; Bacillus; Brevibacterium; Candida; and Hansenula.

Examples of yeast include one or more of Acidomonas, Candida, Hansenula, Komagataella, Ogataea, Pichia, Rhodotorula, Torulopsis. For example, Candida utilis, Hansenula polymorpha, Pichia pastoris, Pichia methanolica, Candida boidinii, Ogataea polymorpha, Acidomonas methanolica, Candida tropicalis, Candida methylica, Candida pseudotropicalis, Candida freyschussii, Candida lipolytica and Candida sonorensis.

Examples of bacteria include one or more of Acidomonas, Albibacter, Amycolatopsis, Ancylobacter, Arthrobacter, Bacillus, Chenggangzhangella, Clostridium, Hydrogenovibrio, Hyphomicrobium, Labrys, Leisingera, Methanococcoides, Methanothermobacter, Methylacidiphilum, Methylibium, Methylobacillus, Methylobacterium, Methylorubrum, Methylocaldum, Methylocapsa, Methyloceanibacter, Methylocella, Methylococcus, Methylocystis, Methyloferula, Methylogaea, Methyloglobulus, Methylohalobius, Methylohalomonas, Methylomicrobium, Methylomonas, Methylonatrum, Methylophaga, Methylophilus, Methylopila, Methylorosula, Methylorubrum, Methylosarcina, Methylosinus, Methylosoma, Methylosphaera, Methylotenera, Methylothermus, Methyloversatilis, Methylovirgula, Methylovorus, Microvirga, Nocardia, Novimethylophilus, Oharaeibacter, Paracoccus, Pontibaca, Pseudomonas, Pyrinomonas, Rhodoblastus, Rhodococcus, Rubrivivax, Shinella, Sporomusa.

Examples of archaea include Methanohalophilus, Methanolacinia, Methanolinea, Methanolobus, Methanomethylovorans, Methanosarcina, Methanobrevibacter, Methanothermococcus, Methanococcus, Methanotorris, Methanothermobacter Methanofervidicoccus, Methanosphaera, and/or Methanimicrococcus.

The methylotrophic microorganism may be selected from one or more of a yeast a bacteria and/or archaea or may be combination of one or more of these.

The methylotrophic microorganism may be a strain of Methylophilus methylotrophus (previously known as Pseudomonas methylotropha), the characteristics of which species are described in UK Pat. No. 1370892, U.S. Pat. No. 4,317,843A, and EP0088530B1 which are incorporated herein.

The methylotrophic microorganism may be Methylophilus methylotrophus DSM 5691.

The microorganism may be a microorganism that has been genetically modified to produce a recombinant methylotrophic microorganism. For example, the microorganisms may be a genetically modified microorganism selected from may include one or more members of the genera Clostridium, Rhodobacter, Xanthobacter, Brevibacterium, Mycobacterium, Amycolaptosis, Bacillus, Methylobacillus, Methylomicrobium, Methylotenera, Paracoccus, Methylocella, Methylacidiphium, Methylobacterium, Methylococcus, Methylobacter, Methylibium, Leisingera, Methylophilus, Methylosulfonomonas, Hyphomicrobium, Methylocystis, Methylosinus, Methylomirabilis, Methylophaga and Methyloversatilis. For example, a microorganisms as described in WO2016019249A1 which is incorporated herein by reference. In some examples, the microorganism is or comprises Methylophilus methylotrophus NCIMB10515. In some examples, the microorganism is or comprises DSM 46235, AS1, ATCC 53528, BCRC 15784, CCUG 58724, KCTC 1759, NRRL B 5352-5364, FRI 1215-1227 or LMG 6787. In some examples, the microorganism is or comprises Methylophilus methylotrophus DSM 5691.

### Culturing

The methods described herein include culturing a methylotrophic microorganism. The microorganism may be cultured using a liquid culture method.

For example, the microorganism may be cultured using a batch culturing process. Batch cultures are easy to operate (a closed system where cells and all the substrates are put in at the beginning of the culturing and no more addition during the process).

The microorganism may be cultured using a fed-batch culture method which allows the addition of substrates during culturing. Fed-batch culture refers to a cell culture method in which additional components are provided to the culture at some point after the start of the culture process.

The microorganism may be cultured using a two-stage cultivation method such as two stage culturing methods. For example, the microorganisms may first be cultured in a batch method and then subsequently cultured using a fed-batch method.

General conditions for the culturing of methylotrophic microorganisms are well known in the field. For example, see U.S. Pat. Nos. 4,306,026A, 3,989,594A, 4,317,843A, WO2017165244A1, WO2011056183A1, WO2011139804A2, WO2013148348A1, WO2014145194A2, WO2017165244A1, WO2018144965A1 and 9,157,058B2 which are all incorporated herein by reference.

The liquid cultures used to grow microorganisms described herein can be housed in culture vessels known and used in the art. Large scale production in a bioreactor vessel can be used to produce large quantities of biomass. The term “bioreactor” or “fermenter” refers to a closed or partially closed vessel in which cells are grown and maintained. The cells may be, but are not necessarily, held in liquid suspension. Rather than being held in liquid suspension, cells may alternatively be grown and/or maintained in contact with, on, or within another non-liquid substrate including but not limited to a solid growth support material.

Bioreactor vessels may be used to contain, isolate, and/or protect the culture environment. The culture vessels include those that are known to those of ordinary skill in the art of large scale microbial culturing. Such culture vessels include but are not limited to one or more of the following: airlift reactors; biological scrubber columns; bubble columns; stirred tank reactors; continuous stirred tank reactors; counter-current, upflow, expanded-bed reactors; digesters and digester systems, for example, such known in the art of bioremediation; filters including but not limited to trickling filters, rotating biological contactor filters, rotating discs, soil filters; fluidized bed reactors; gas lift fermenters; immobilized cell reactors; loop reactors; membrane biofilm reactors; pachuca tanks; packed-bed reactors; plug-flow reactors; static mixers; trickle bed reactors; and/or vertical shaft bioreactors.

Microbial culturing aimed at the commercial production of biomass e.g., protein product as described herein, such as single cell protein may be performed in bioreactors at large scale (e.g., 500 L; 1,000 L; 5,000 L; 10,000 L; 50,000 L; 100,000 L; 1,000,000 L bioreactor volumes and higher).

A bioreactor containing nutrient medium may be inoculated with production cells (of the methylotrophic microorganism). Generally, there will follow a lag phase prior to the cells beginning to double. After the lag phase, the cell doubling time decreases and the culture goes into the logarithmic phase. The logarithmic phase is eventually followed by an increase of the doubling time that, while not intending to be limited by theory, is thought to result from either a mass transfer limitation, depletion of nutrients including nitrogen or mineral sources, or a rise in the concentration of inhibitory chemicals, or quorum sensing by the microbes. The growth slows down and then ceases when the culture enters the stationary phase. There may be an arithmetic growth phase preceding the stationary phase. In order to harvest cell mass (i.e. biomass), the cultured cells are harvested in the logarithmic phase and/or in the arithmetic phase and/or in the stationary phase.

The bioreactor or fermenter is used to culture cells through the various phases of their physiological cycle. A bioreactor is utilized for the cultivation of cells, which may be maintained at particular phases in their growth curve. The use of bioreactors is advantageous in many ways for cultivating chemoautotrophic growth. For certain embodiments, protein-rich biomass, which is used to produce single cell protein, is grown to high densities in liquid suspension. Generally, the control of growth conditions, including control of dissolved carbon dioxide, oxygen, and other gases such as hydrogen, as well as other dissolved nutrients, trace elements, temperature and pH, is facilitated in a bioreactor. For certain embodiments, protein-rich biomass, which is used to produce amino acids, peptides, proteins, hydrolysates, extracts, or whole cell products, is grown to high densities and/or grown at high productivities, in liquid suspension within a bioreactor.

In certain examples, one or more gases are dissolved into the culture medium, including but not limited to gaseous electron donors such as, but not limited to, one or more of the following: hydrogen, carbon monoxide, methane, hydrogen sulphide or other sour gases; gaseous carbon sources such as, but not limited to one or more of the following: CO₂, CO, CH₄; and electron acceptors such as, but not limited to, oxygen, either within air (e.g., 20.9% oxygen) or as pure O₂ or as an O₂-enriched gas. The dissolution of these and other gases into solution may be achieved using a system of compressors, flowmeters, and flow valves known to one skilled in the art of fermentation engineering, that feed into one of more of the following widely used systems for dispersing gas into solution: sparging equipment; diffusers including but not limited to dome, tubular, disc, or doughnut geometries; coarse or fine bubble aerators; venturi equipment. Surface aeration and/or gas mass transfer may also be achieved using paddle aerators and the like. Gas dissolution may be enhanced by mechanical mixing with an impeller or turbine, as well as hydraulic shear devices to reduce bubble size. Following passage through the reactor system holding microorganisms which uptake the gases, the residual gases may either be recirculated back to the bioreactor, burned for process heat, flared, injected underground, or released into the atmosphere.

A culture medium for culturing (i.e. growth and production) may be used, comprising an aqueous solution containing suitable minerals, salts, vitamins, cofactors, buffers, and other components needed for microbial growth, known to those skilled in the art For example, see “Bailey and Ollis, Biochemical Engineering Fundamentals, 2nd ed; pp 383-384 and 620-622; McGraw-Hill: New York (1986)”.

The chemicals used for maintenance and growth of microbial cultures as known in the art may be included in the culture media. These chemicals may include but are not limited to one or more of the following: nitrogen sources such as ammonia, ammonium (e.g., ammonium chloride (NH₄Cl), ammonium sulfate ((NH₄)₂SO₄)), nitrate (e.g., potassium nitrate (KNO₃)), urea or an organic nitrogen source; phosphate (e.g., disodium phosphate (Na₂HPO₄), potassium phosphate (KH₂PO₄), phosphoric acid (H₃PO₄), potassium dithiophosphate (K₃PS₂O₂), potassium orthophosphate (K₃PO₄), dipotassium phosphate (K₂HPO₄)); sulfate; yeast extract; chelated iron; potassium (e.g., potassium phosphate (KH₂PO₄), potassium nitrate (KNO₃), potassium iodide (KI), potassium bromide (KBr)); and other inorganic salts, minerals, and trace nutrients (e.g., sodium chloride (NaCl), magnesium sulfate (MgSO₄7H₂O) or magnesium chloride (MgCl), calcium chloride (CaCl) or calcium carbonate (CaCCb), manganese sulfate (MnSO₄7H₂O) or manganese chloride (MnCl), ferric chloride (FeCl), ferrous sulfate (FeSO₄7H₂O) or ferrous chloride, sodium bicarbonate (or sodium carbonate (Na₂CO₃), zinc sulfate (ZnSO₄) or zinc chloride (ZnC), ammonium molybdate (NH₄MoO₄) or sodium molybdate (Na₂MoO₄2H₂O), cuprous sulfate (CUSO₄) or copper chloride, cobalt chloride, aluminum chloride, lithium chloride (LiCl), boric acid, nickel chloride, tin chloride, barium chloride, copper selenate or sodium selenite, sodium metavanadate, chromium salts.

Microorganisms described herein can be cultured in media of any type (rich or minimal), including fermentation medium, and any composition. As would be understood by one of ordinary skill in the art, routine optimization would allow for use of a variety of types of media. The selected medium can be supplemented with various additional components. Some non-limiting examples of supplemental components include glucose, fructose, sucrose, starches, polysaccharides, protein hydrolysates, antibiotics, IPTG for gene induction, and ATCC Trace Mineral Supplement. Similarly, other aspects of the medium and growth conditions of the microorganisms described herein may be optimized through routine experimentation.

In some examples, the concentrations of nutrient chemicals (e.g., electron donors, electron acceptors, carbon sources, and/or various mineral nutrients), are maintained within the bioreactor close to or at their respective optimal levels for optimal carbon uptake, fixation, conversion, and/or production of biomass, and in particular protein, which varies depending upon the microorganism utilized but may be routinely determined and/or optimized by one of ordinary skill in the art of culturing microorganisms.

In some examples, the culture medium may comprise potassium dihydrogen phosphate (KH₂PO₄). In some examples, the culture medium may comprise sodium phosphate dibasic dodecahydrate (Na₂HPO₄·12H₂O) or disodium phosphate. In some examples, the culture medium may comprise ammonium sulphate ((NH₄)₂SO₄). In some examples, the culture medium may comprise magnesium sulphate heptahydrate (MgSO₄·7H₂O). In some examples, the culture medium may comprise a trace elements solution. In some examples, the culture medium comprises potassium dihydrogen phosphate, sodium phosphate dibasic dodecahydrate or disodium phosphate, ammonium sulphate and/or magnesium sulphate heptahydrate. In some examples, the culture medium comprises potassium dihydrogen phosphate, sodium phosphate dibasic dodecahydrate or disodium phosphate, ammonium sulphate, magnesium sulphate heptahydrate and/or a trace elements solution.

In some examples, the culture medium comprises a concentration of potassium dihydrogen phosphate from about 1 to 2 g/L. For example, 1.305 g/L.

In some examples, the culture medium comprises a concentration of comprise sodium phosphate dibasic dodecahydrate from about 2 to 3 g/L. For example, 2.13 g/L. In some examples, the culture medium comprises a concentration of comprise disodium phosphate from about 4 to 6 g/L. For example, 5.37 g/L.

In some examples, the culture medium comprises a concentration of ammonium sulphate from about 1 to 2 g/L. For example, 1.5 g/L.

In some examples, the culture medium comprises a concentration of magnesium sulphate heptahydrate from about 0 to 0.6 g/L. For example, 0.45 g/L.

The trace elements solution may comprise one or more of iron (II) sulfate heptahydrate, calcium chloride dihydrate, manganese (II) sulfate, zinc sulfate heptahydrate, sodium molybdate dihydrate, copper (II) sulphate pentahydrate, cobalt (II) chloride hexahydrate, and/or boric acid. Therefore, the trac element solution may be a solution that includes a source of one or more of iron, calcium, manganese, zinc, molybdenum, copper, cobalt and boron.

In some examples, the trace elements solution comprises iron (II) sulfate heptahydrate at a concentration from 0.05 to 2.5 g/L. For example, 0.02 g/L.

In some examples, the trace elements solution comprises calcium chloride dihydrate at a concentration from 0.05 to 2.5 g/L. For example, 0.02 g/L.

In some examples, the trace elements solution comprises manganese (II) sulfate at a concentration from 0.002 to 0.006 g/L. For example, 0.0049 g/L.

In some examples, the trace elements solution comprises zinc sulfate heptahydrate at a concentration from 0.001 to 0.004 g/L. For example, 0.0026 g/L.

In some examples, the trace elements solution comprises sodium molybdate dihydrate at a concentration from 0.0005 to 0.001 g/L. For example, 0.0008 g/L.

In some examples, the trace elements solution comprises copper (Il) sulphate pentahydrate at a concentration from 0.0005 to 0.001 g/L. For example, 0.0008 g/L.

In some examples, the trace elements solution comprises cobalt (II) chloride hexahydrate at a concentration from 0.0005 to 0.001 g/L. For example, 0.0008 g/L.

In some examples, the trace elements solution comprises boric at a concentration from 0.0004 to 0.001 g/L. For example, 0.0006 g/L.

In some examples, the culture medium may comprise an acid mineral solution comprising the components as defined in Table A below:

TABLE A
Acid Mineral Solution (AMS)

Volume

| 1000 mL | 500 mL | Component |
|---|---|---|
| 270 | 135 | g of H3PO4 |
| 100 | 50 | g of MgSO4•7H20 |
| 2 | 1 | g of FeSO4•7 H20 |
| 2 | 1 | g of CaCl2•2 H20 |
| 0.5 | 0.25 | g of MnSO4•H20 |
| 0.26 | 0.13 | g of ZnSO4•7 H20 |
| 0.08 | 0.04 | g of Na2MoO4•2H2O |
| 0.08 | 0.04 | g of CuCl2•2 H20 |
| 0.08 | 0.04 | g of CoCl2•6 H20 |
| 0.06 | 0.03 | g of H3BO3 |

One or more of the following parameters may be monitored and/or controlled in the bioreactor: waste product levels; pH; temperature; salinity; dissolved oxygen; dissolved carbon dioxide gas; liquid flow rates; agitation rate; gas pressure. The operating parameters may be monitored with sensors (e.g., dissolved oxygen probe or oxidation-reduction probe to gauge electron donor/acceptor concentrations), and/or are controlled either manually or automatically based upon feedback from sensors through the use of equipment including but not limited to actuating valves, pumps, and agitators. The temperature of the incoming culture medium as well as of incoming gases may be regulated by systems such as, but not limited to, coolers, heaters, and/or heat exchangers.

By controlling different aspects of the culturing conditions, and exposing the culturing microorganism to these conditions, the biomass and subsequent products derived therefrom (e.g. single cell protein) have desired and advantageous properties, such as the properties described herein.

For example, by controlling gas flow rate. Gas flow rate is the volume of gas that passes a particular point in a particular period of time.

For example, by controlling the stirring rate.

For example, by controlling pH of the culture medium.

For example, by controlling temperature of the culture medium.

For example, by controlling methanol concentration in the culture medium.

For example, by controlling methanol feed rate.

For example, by controlling nitrogen concentration in the culture medium.

For example, by controlling nitrogen feed rate.

For example, by controlling nutrient concentration.

For example, by controlling nutrient feed rate.

For example, by controlling dissolved oxygen concentration.

For example, by controlling phosphorus concentration.

For example, by controlling magnesium concentration.

The culture conditions may be selected or configured to induce the microorganism to ferment the methanol present in the culture medium. For example, any one or more gas flow rate; stirring rate; pH; temperature; methanol concentration; methanol feed rate; nitrogen concentration; nitrogen feed rate; nutrient concentration; magnesium concentration; dissolved oxygen concentration; phosphorus concentration and/or nutrient feed rate may be selected to provide for fermentation of the methanol.

Culture conditions may be defined by reference to a concentration or rate. It will be understood by the skilled person that a concentration in a culture medium or vessel may refer to a continuous concentration or may refer to a concentration as taken at a specific time point during culturing. It will also be understood that rates refers of components and/or conditions may be scale dependent. For example, dependent of the volume of the culture medium and/or culture vessel. A such rates may be defined as a volume of feed per unit of volume of reactor per unit of time (i.e. vvm). In other examples, rates may be defined relative to the amount of biomass produced, for example volume of component (e.g., methanol) per unit of time per unit cell mass

It has been found that culturing the microorganism under such conditions leads to the production of protein within the microorganism (i.e. in the biomass cultured) as well in the culture medium (after culturing referred to as culture broth) that has desirable and tuneable properties particularly suited for use in a variety of different food products. The amount of protein produced may be relatively high in comparison to culturing under other conditions.

Some or all of the conditions abovementioned (i.e. at least one) may be altered or changed (for example, increased or decreased) throughout the time course of the culturing. In some examples, some or all of the culturing conditions (i.e. at least one) may be kept constant throughout the time course of the culturing.

The culturing conditions may be selected or configured to induce the microorganisms to produce protein (either in the biomass and/or the culture broth) that has specific properties. The culturing conditions may be selected or configured to induce the microorganisms to produce protein, biomass and/or culture broth (i.e. a single cell protein product) that has specific properties.

For example, by changing one or more of the above mentioned conditions, the single cell protein product (protein, biomass or culture broth) produced may have properties that are suited for use of the single cell protein product (e.g. protein, biomass and/or culture broth) in dairy analogues. For example, by changing one or more of the above mentioned conditions, the single cell protein product (protein, biomass or culture broth) produced may have properties that are suited for use of the single cell protein product (protein, biomass and/or culture broth) in meat analogues. For example, by changing one or more of the above mentioned conditions, the single cell protein product (protein, biomass or culture broth) produced may have properties that are suited for use of the single cell protein product (protein, biomass and/or culture broth) in egg analogues.

In some examples, the properties imparted on the single cell protein product (protein, biomass and/or culture broth) may provide a single cell protein product (e.g. biomass or culture broth) that may be used in a food product without further processing.

In some examples, the properties imparted on the single cell protein product (protein, biomass and/or culture broth) may be one or more of rheological properties, protein concentration, aesthetic properties, organoleptic properties, structural properties and/or physical properties. For example, a fibrous structure and a colour that is mimetic of uncooked animal meat. For example, pink or red.

These properties may have certain advantages in different food products. For example, a dairy analogue where the single cell protein product (protein of the biomass and/or culture broth or the biomass) comprise a relatively low viscosity, a cream like texture and/or a white or off white colour. For example, a meat analogue where the single cell protein product (protein of the biomass and/or culture broth or the biomass) comprise a relatively low viscosity, a relatively high oil holding capacity (OHC), organoleptic properties mimetic of meat and/or appearance mimetic of meat. For example, a low viscosity may be equal to or less than 200 mPa·s. For example, a low viscosity may be equal to or less than 50 mPa·s. For example, a high viscosity may be equal to or more than 75 mPa·s. For example, a high viscosity may be equal to or more than 300 mPa·s.

In some examples, the culturing conditions may include stress conditions. That is to say the microorganisms may be cultured under one or more stress conditions for at least a period of time of the culturing.

In some examples, the stress conditions include gas and/or metabolite limiting conditions. For example, culturing in oxygen limiting condition. Oxygen limitation refers to reducing the oxygen concentration in the aqueous medium or atmosphere in contact with the cells. For example, oxygen limitation refers to at a level (e.g., concentration) of oxygen available to the microorganism which is below the level needed for normal microorganism metabolism, growth, reproduction and/or viability.

For example, culturing in nitrogen limiting conditions. Nitrogen limiting conditions refers to growth conditions which include a level (e.g., concentration) of nitrogen (e.g., ammonium or nitrate) available to the microorganism which is below the level needed for normal microorganism metabolism, growth, reproduction and/or viability.

In some examples, the microorganism may be cultured using restricted and/or unrestricted culturing processes. For examples, cultured using only a restricted culturing process. In other examples, cultured using only an unrestricted culturing process. In some examples, first cultured using only an unrestricted or restricted culturing process and then cultured using a culturing process that differs from the first culturing process. For examples, first culturing using a restricted culturing process then cultured using an unrestricted culturing process. Alternatively, first culturing using an unrestricted culturing process then cultured using a restricted culturing process.

In some examples, the culture condition may be dissolved oxygen concentration. In some examples, the dissolved oxygen concentration may be at least 5%. In some examples, the dissolved oxygen concentration may be at least 15%. In some examples, the dissolved oxygen concentration may be at least 30%. In some examples, the dissolved oxygen concentration may be at least 40%. In some examples, the dissolved oxygen concentration may be at least 45%. In some examples, the dissolved oxygen concentration may be at least 50%. In some examples, the dissolved oxygen concentration may be from 5% to 50%. For example, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50%. For example, the dissolved oxygen concentration may be from 15% to 50%. For example, the dissolved oxygen concentration may be from 15% to 35%. In some examples, the dissolved oxygen concentration may be from 15% to 45%. For example, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45%. For example, the dissolved oxygen concentration may be from 0% to 35%. For example, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35%. For example, the dissolved oxygen concentration may be from 15% to 30%. For example, 15, 16, 17, 18, 19, 20, 21, 22, 23, 24, 25, 26, 27, 28, 29 or 30%. In some examples, the dissolved oxygen concentration may be from 0 to 5%. In some examples, the dissolved oxygen concentration may be from 20 to 25%. The dissolved oxygen concentration may be selected depending on the desired final product and the food product for which the single cell protein product may be incorporated into. In some examples, the dissolved oxygen concentration may be 5%. In some examples, the dissolved oxygen concentration may be 15%. In some examples, the dissolved oxygen concentration may be about 30%. In some examples, the dissolved oxygen concentration may be about 45%. In some examples, the dissolved oxygen concentration may be 50%. Without being bound by theory, a dissolved oxygen concentration of about 30% may provide an improved yield of cells and ergo an improved yield of single cell protein product. Without being bound by theory, a dissolved oxygen concentration of about 50% may provide an improved yield of cytochrome C in a single cell protein product.

In some examples, where the method is a method as described herein which leads to the production of relatively high level of cytochrome C, the dissolved oxygen concentration may be about 15% to 60%. In some examples, where the method is a method as described herein which leads to the production of relatively high level of cytochrome C, the dissolved oxygen concentration may be about 15% to 50%. In some examples, where the method is a method as described herein which leads to the production of relatively high level of cytochrome C the dissolved oxygen concentration may be about 15% to 50%. In some examples, where the method is a method as described herein which leads to the production of relatively high level of cytochrome C the dissolved oxygen concentration may be about 15%. In some examples, where the method is a method as described herein which leads to the production of relatively high level of cytochrome C the dissolved oxygen concentration may be about 25%. In some examples, where the method is a method as described herein which leads to the production of relatively high level of cytochrome C the dissolved oxygen concentration may be about 30%. In some examples, where the method is a method as described herein which leads to the production of relatively high level of cytochrome C the dissolved oxygen concentration may be about 50%.

In some examples, the culture condition may be aeration rate. Aeration rate refers to the rate at which oxygen is introduced, and is typically recorded in volume (e.g. m³) gas per volume (m³) reactor volume per unit of time (i.e. per hour). Aeration may be achieved by introduction of air or using oxygen in other forms, such as pure or mixed with other gases. The aeration rate may be at least 0.1 vvm (i.e. volume of gas/volume of reactor or vessal/minute). In some examples, the aeration rate is at least 1.5 vvm. In some examples, the aeration rate is from 0.1 and 3 vvm. In some examples, the aeration rate is from 1.5 to 3 vvm. For example, at least 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3 vvm. For example, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 vvm. For example 1.5, 1.6, 1.7, 1.8, 1.9, 2, 2.1, 2.2, 2.3, 2.4, 2.5, 2.6, 2.7, 2.8, 2.9, or 3 vvm.

In some examples, the methanol concentration is from 0 to 10 g/L. In some examples, 0 to 6 g/L. In some examples, 0.1 to 4 g/L. For example, 0.0, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 g/L.

In some examples, the methanol feed rate may be at least 2 g/kg/hour. In some examples, the methanol feed rate may be at least 4 g/kg/hour. In some examples, the methanol feed rate may be at least 8 g/kg/hour. In some examples, the methanol feed rate may be at least 20 g/kg/hour. In some examples, the methanol feed rate may be at least 30 g/kg/hour. In some examples, the methanol feed rate may be at most 20 g/kg/hour. In some examples, the methanol feed rate may be at most 30 g/kg/hour. In some examples, the methanol feed rate may be from 2 to 20 g/kg/hour. In some examples, the methanol feed rate may be from 4 to 20 g/kg/hour. For example, 4, 5, 6, 7, 8, 9, 10, 11, 12, 13, 14, 15, 16, 17, 18, 19, 20, 21, 22, 23, 24, 25, 26, 27, 28, 29, or 30 g/kg/hour. In some examples, the methanol feed rate may be from 4 to 8 g/kg/hour. For example, 4, 5, 6, 7 or 8 g/kg/hour.

In some examples, wherein the method produces a relatively high concentration of cytochrome C the methanol feed rate may be about 3 to 12 g/kg/hour. In some examples, wherein the method produces a relatively high concentration of cytochrome C the methanol feed rate may be about 4 to 16 g/kg/hour. In some examples, wherein the method produces a relatively high concentration of cytochrome C the methanol feed rate may be about 4 to 20 g/kg/hour. In some examples, wherein the method produces a relatively high concentration of cytochrome C the methanol feed rate may be about 4 to 30 g/kg/hour

In some examples, the methanol feed rate may be dynamic. For example, the methanol feed rate may change throughout the culturing process. For example, the methanol feed rate may increase or decrease during culturing. For example, the methanol feed rate may increase during culturing.

In some examples, the methanol feed rate may increase from 4 g/kg/h to 8 g/kg/h. In some examples, the methanol feed rate may increase from 4 g/kg/h to 20 g/kg/h. In some examples, the methanol feed rate may increase from 3 g/kg/h to 12 g/kg/h. In some examples, the methanol feed rate may increase from 4 g/kg/h to 16 g/kg/h.

In some examples, the methanol feed rate may increase from 4 g/kg/h to 8 g/kg/h and the dissolved oxygen concentration may be 5%. In some examples, the methanol feed rate may increase from 4 g/kg/h to 20 g/kg/h and the dissolved oxygen concentration may be 5%. In some examples, the methanol feed rate may increase from 4 g/kg/h to 8 g/kg/h and the dissolved oxygen concentration may be 50%. In some examples, the methanol feed rate may increase from 4 g/kg/h to 20 g/kg/h and the dissolved oxygen concentration may be 50%.

In some examples, the methanol feed rate may be about 2 g/kg/hour. In some examples, the methanol feed rate may be about 2 g/kg/hour and the dissolved oxygen concentration may be 15%.

In some examples, wherein the method produces a relatively high concentration of cytochrome C the methanol feed rate may increase from 3 to 12 g/kg/hour throughout the duration of culturing. In some examples, wherein the method produces a relatively high concentration of cytochrome C the methanol feed rate may increase from 4 to 16 g/kg/hour throughout the duration of culturing. In some examples, the methanol feed rate may increase from 3 g/kg/h to 12 g/kg/h and the dissolved oxygen concentration may be 5%. In some examples, the methanol feed rate may increase from 3 g/kg/h to 12 g/kg/h and the dissolved oxygen concentration may be 30%. In some examples, the methanol feed rate may increase from 3 g/kg/h to 12 g/kg/h and the dissolved oxygen concentration may be 50%. In some examples, the methanol feed rate may increase from 4 g/kg/h to 16 g/kg/h and the dissolved oxygen concentration may be 30%.

In some examples, the methanol feed may be diluted methanol. For example, the feed may include, 20%, 30%, 40%, or about 50% methanol.

In some examples, the temperature is from 20 to 70° C. For example, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70° C. In some examples, the temperature is from 22 to 55° C. In some examples, the temperature is from 30 to 37° C. For example, 30, 31, 32, 33, 34, 35, 36 or 37° C.

In some examples, nitrogen concentration is from 0 to 1.2 g/L. For example, 0, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.35, 0.4, 0.45, 0.5, 0.55, 0.6, 0.65, 0.7, 0.75, 0.8, 0.85, 0.9, 0.95, 1, 1.05, 1.1, 1.15, 1.2 g/L. In some examples, nitrogen concentration is from 0 to 0.3 g/L. For example, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3 g/L.

In some examples, the pH is from 5 to 7. For example, 5, 6 or 7. In some examples, the pH is from 5.9 to 6.8. For example, 5.9, 6, 6.1, 6.2, 6.3, 6.4, 6.5, 6.6, 6.7 or 6.8. In some examples, the pH is 6 to 6.8. For example, 6, 6.1, 6.2, 6.3, 6.4, 6.5, 6.6, 6.7, or 6.8.

In some examples, the phosphorus concentration is from 0 to 2.0 g/l. For example, 0, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.35, 0.4, 0.45, 0.5, 0.55, 0.6, 0.65, 0.7, 0.75, 0.8, 0.85, 0.9, 0.95, 1, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, or 2 g/L. In some examples, the phosphorus concentration is from 0.08 g/L. For example, 0, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.35, 0.4, 0.45, 0.5, 0.55, 0.6, 0.65, 0.7, 0.75, or 0.8 g/L.

In some examples, magnesium concentration is from 0 to 80 mg/l. For example, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80 g/L. In some examples, the magnesium concentration is from 0 to 40 g/L. For example, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 g/L.

Restricted culture process refers to culturing the microorganism under conditions that are not optimal conditions for growth (for example culturing with stressors).

Unrestricted culture process refers to culturing the microorganism under conditions that are optimal conditions for growth (for example culturing without any stressors).

In some examples, the cultivation or culturing may be a batch fermentation process. “Fermentation” refers to the cultivation of a microorganism in which sugar is converted to alcohol and is independent of aeration regime.

The term “batch mode” or “batch fermentation” refers to a culture mode wherein the cells are cultured in the initially present fermentation (culture) medium without any change in medium composition or the volume of the medium. Thus, in batch mode no substantial or significant amount of fresh liquid culture medium is added to the cell culture and no substantial or significant amount of liquid culture medium is removed from the cell culture during culturing.

The use of a batch process may provide a single cell protein product (e.g. biomass) that has a relatively low viscosity. For example, a low viscosity may be equal to or less than 200 mPa. For example, a low viscosity may be equal to or less than 50 mPa·s. The use of a batch fed process may provide a single cell protein product that has an appearance that is similar to a dairy product. For example, has a creamy texture and/or a white or off white colour.

The use of a batch process may provide a single cell protein product that has properties that are suited for use of the single cell protein product as a dairy analogue, an emulsifier and/or a foaming agent. For example, appearance, texture, rheological properties and/or organoleptic properties.

In some examples, the cultivation or culturing may be a fed-batch fermentation process. The term “fed-batch mode” or “fed-batch fermentation” refers to a culture mode wherein the cells are cultured in the initially present fermentation medium and a feed solution is added in a periodic or continuous manner without substantial or significant removal of liquid culture medium during culturing. Fed-batch cultures can include various feeding regimens and times, for example, daily feeding, feeding more than once per day, or feeding less than once per day, and so on.

The use of a fed-batch process may provide a single cell protein product that has a relatively high viscosity. For example, a high viscosity may be more than 50 mPa·s. For example, more than 75 mPa·s. For example, a high viscosity may be more than 300 mPa·s. The use of a fed-batch process may provide a single cell protein product that has an appearance that is similar to a meat (e.g. appearance similar to a meat analogue or animal derived meat product).

The use of a fed-batch process may provide a single cell protein product that has properties that are suited for use of the single cell protein product as a meat analogue. For example, appearance, texture, rheological properties and/or organoleptic properties.

In some examples, the microorganism may be cultured using a continuous culturing method. “Continuous” culturing refers to the method of culturing wherein a volume of cells and media are removed from the culture, cells are harvested, and new media replaces what was removed.

In some examples, the culturing may be carried out using a sequential batch culturing process. Sequential batch culturing refers to culturing that includes replenishing the medium after end-of-the-batch, so that a new batch can start using the produced biomass as inoculum.

In some examples, the culturing process may be a feed-draw process. This refers to a fed-batch culturing process where at a certain time point part of the broth is removed so that the fed-batch can be extended.

In some examples culturing may include cell retention culturing methods. This refers to culturing where the cultured cells or biomass is maintained in the culture vessel and only the culture broth is removed.

In some examples, the culturing process may be a cell-recycle fermentation process. Cell recycle fermentation refers to a process in which cultured cells (i.e. biomass) is partially or wholly recycled by means of centrifugation, filtration or gravity sedimentation.

In some examples, culturing may include a combination of any of the culturing processes described herein.

In some examples, the method of culturing may lead to a biomass of cultured microorganism having a relatively high concentration of cytochrome C or single cell protein product derived therefrom (e.g. at least 200 pmol/mg dry weight of microorganism or biomass thereof). In some examples, the method of culturing may lead to a culture broth e.g. cell culture media and cultured cells having a relatively high concentration of cytochrome C (e.g. at least 10×10⁻³ mM), or single cell protein product derived therefrom e.g. harvested or processed culture broth having a relatively high concentration of cytochrome C (e.g. at least 10×10⁻³ mM) due to the culturing conditions used.

For example, culturing methods described herein may lead to a microorganism producing higher than normal levels of cytochrome C. For example, higher than when using standard or prior art culturing methods. As such, there is also provided a microorganism that includes or produces a concentration of cytochrome C of at least 200 pmol/mg of dry weight of microorganism or biomass thereof.

In particular, wherein the method comprises producing a relatively high concentration of cytochrome C the microorganism is or comprises DSM 46235, AS1, ATCC 53528, BCRC 15784, CCUG 58724, KCTC 1759, NRRL B 5352-5364, FRI 1215-1227 or LMG 6787. In some examples, wherein the method comprises producing a relatively high concentration of cytochrome C the microorganism is or comprises Methylophilus methylotrophus DSM 5691. In some examples, the microorganism is or comprises Methylophilus methylotrophus NCIMB10515.

Methods of determining the concentration of cytochrome C will be known by those skilled in the art. For example, concentration of cytochrome C may be determined using spectrophotometric analysis. In some examples, the concentration of cytochrome C may be a normalised concentration. For example, normalised to the dry mass of an original sample.

Also provided in some examples, is a method of producing a single cell protein product that comprises a concentration of cytochrome C of at least 10×10⁻³ mM. The single cell protein product may be the culture broth (e.g. cultured cells and culture medium). The single cell protein product may be a product derived from the culture broth.

As such, also provided herein in is a method of producing a culture broth comprising at least 10×10⁻³ mM cytochrome C. In some examples, the method comprises culturing the methylotrophic organism at dissolved oxygen concentration of 5% and with a increasing methanol feed rate starting from 3 g/kg/h and ending at 12/kg/hr. In some examples, the culture broth (including cultured cells and culture medium) is centrifuged to provide a supernatant and pellet. In some examples, the supernatant is the single cell protein product comprising the at least 10×10⁻³ mM cytochrome C. In some examples, the supernatant collected is the single cell protein product and comprises at least or about 17 ×10⁻³ mM of cytochrome C. In some examples, the biomass (pellet from centrifugation) comprises a cytochrome C concentration of at least or about 330 μmol/mg of biomass.

In some examples, the method comprises culturing the methylotrophic organism at dissolved oxygen concentration of 30% and with a increasing methanol feed rate starting from 3 g/kg/h and ending at 12/kg/hr. In some examples, the culture broth (including cultured cells and culture medium) is centrifuged to provide a supernatant and pellet. In some examples, the supernatant is the single cell protein product comprising the at least 10×10⁻³ mM cytochrome C. In some examples, the supernatant collected is the single cell protein product and comprises at least or about 20 ×10⁻³ mM of cytochrome C. In some examples, the biomass (pellet from centrifugation) comprises a cytochrome C concentration of at least or about 420 μmol/mg of biomass

In some examples, the method comprises culturing the methylotrophic organism at dissolved oxygen concentration of 50% and with a increasing methanol feed rate starting from 3 g/kg/h and ending at 12/kg/hr. In some examples, the culture broth (including cultured cells and culture medium) is centrifuged to provide a supernatant and pellet. In some examples, the supernatant is the single cell protein product comprising the at least 10×10⁻³ mM cytochrome C. In some examples, the supernatant collected is the single cell protein product and comprises at least or about 27 ×10⁻³ mM of cytochrome C. In some examples, the biomass (pellet from centrifugation) comprises a cytochrome C concentration of at least or about 410 μmol/mg of biomass.

In some examples, the method comprises culturing the methylotrophic organism at dissolved oxygen concentration of 30% and with a increasing methanol feed rate starting from 4 g/kg/h and ending at 16/kg/hr. In some examples, the culture broth (including cultured cells and culture medium) is centrifuged to provide a supernatant and pellet. In some examples, the supernatant is the single cell protein product comprising the at least 10×10⁻³ mM cytochrome C. In some examples, the supernatant collected is the single cell protein product and comprises at least or about 14 ×10⁻³ mM of cytochrome C. In some examples, the biomass (pellet from centrifugation) comprises a cytochrome C concentration of at least or about 240 μmol/mg of biomass.

In some examples, the single cell protein product is a product derived from the culture broth. For example, the method further comprises at least partially disrupting the cultured cells in the culture media as described herein (for example, using heat, acidification, flocculation, sonification, enzymes and/or homogenisation). The disrupted cells and culture media may then be centrifuged as described herein. For example, centrifuged at a force of at least 2000 g. For example, at a force of about 18,000 g. In some examples, the supernatant collected is the single cell protein product comprising the at least 10×10⁻³ mM cytochrome C.

In some examples, the single cell protein product (e.g. culture broth or product derived therefrom) comprises at least 10, 11, 12, 13, 14, 15, 16, 17, 18, 19, 20, 21, 22, 23, 24, 25, 26, 27, 28, 29 or 30 ×10⁻³ mM cytochrome C. In some examples, the single cell protein product (e.g. culture broth or product derived therefrom) comprises about 10, 11, 12, 13, 14, 15, 16, 17, 18, 19, 20, 21, 22, 23, 24, 25, 26, 27, 28, 29 or 30 ×10⁻³ mM cytochrome C.

In some examples, the method comprises culturing the methylotrophic organism at dissolved oxygen concentration of 5% and with a increasing methanol feed rate starting from 4 g/kg/h and ending at 8 g/kg/hr.

In some examples, wherein the method comprises a method of producing a single cell protein product that has a relatively high concentration of cytochrome C as described herein (e.g. at least or about 10, 11, 12, 13, 14, 15, 16, 17, 18, 19, 20, 21, 22, 23, 24, 25, 26, 27, 28, 29 or 30 ×10⁻³-mM), the concentration of cytochrome C may be reached or produced by culturing for about 24 hours. In some examples, the concentration of cytochrome C reached or produced by culturing for about 48 hours. For example, culturing for up to 48 hours may provide a single cell protein product that has a concentration of cytochrome C of at least or about 10, 11, 12, 13, 14, 15, 16, 17, 18, 19, 20, 21, 22, 23, 24, 25, 26, 27, 28, 29 or 30 ×10⁻³-mM.

The single cell protein product may be harvested and processed using centrifugation as described herein. For example, centrifuged at a force of at least 2000 g. In some examples, at about 18,000 g. The centrifugation pellet may then be freeze dried to provide the single cell protein product. Such a method may produce a single cell protein product (i.e. pellet obtained from centrifugation of culture broth and subsequent drying) that has a water holding capacity of between 3 and 7 grams of water per gram of dry single cell protein product. For example, 5.97 g/g of dry pellet. The single cell protein product may have an oil holding capacity of about 1 to about 4 gram oil per gram of dried single cell protein product. For example, about 1.44 g/g of dry powder. The single cell protein product may have a brown colour. The single cell protein product may have a structuring property with a viscoelastic Modulus of 41.1 Pa at the end of cooling.

In some examples, the method comprises culturing the methylotrophic organism at dissolved oxygen concentration of 5% and with a increasing methanol feed rate starting from 4 g/kg/h and ending at 20 g/kg/hr.

The single cell protein product may be harvested and processed using centrifugation as described herein. For example, centrifuged at a force of at least 2000 g. In some examples, at about 18,000 g. The centrifugation pellet may then be freeze dried to provide the single cell protein product. Such a method may produce a single cell protein product (i.e. pellet obtained from centrifugation of culture broth and subsequent drying) that has a water holding capacity of between 3 and 7 grams of water per gram of dry single cell protein product. For example, 4.7 g/g of dry pellet. The single cell protein product may have an oil holding capacity of about 1 to about 4 gram oil per gram of dried single cell protein product. For example, about 1.73 g/g of dry powder. The single cell protein product may have a brown/green colour. The single cell protein product may have a structuring property with a viscoelastic Modulus of 15.27 Pa at the end of cooling.

In some examples, the method comprises culturing the methylotrophic organism at dissolved oxygen concentration of 50% and with a increasing methanol feed rate starting from 4 g/kg/h and ending at 8/kg/hr.

The single cell protein product may be harvested and processed using centrifugation as described herein. For example, centrifuged at a force of at least 2000 g. In some examples, at about 18,000 g. The centrifugation pellet may then be freeze dried to provide the single cell protein product. Such a method may produce a single cell protein product (i.e. pellet obtained from centrifugation of culture broth and subsequent drying) that has a water holding capacity of between 3 and grams of water per gram of dry single cell protein product. For example, 3.29 g/g of dry pellet. The single cell protein product may have an oil holding capacity of about 1 to about 4 gram oil per gram of dried single cell protein product. For example, about 1.66 g/g of dry powder. The single cell protein product may have a brown/pink colour. The single cell protein product may have a structuring property with a viscoelastic Modulus of 18.29 Pa at the end of cooling.

In some examples, the method comprises culturing the methylotrophic organism at dissolved oxygen concentration of 50% and with a increasing methanol feed rate starting from 4 g/kg/h and ending at 20/kg/hr.

The single cell protein product may be harvested and processed using centrifugation as described herein. For example, centrifuged at a force of at least 2000 g. In some examples, at about 18,000 g. The centrifugation pellet may then be freeze dried to provide the single cell protein product. Such a method may produce a single cell protein product (i.e. pellet obtained from centrifugation of culture broth and subsequent drying) that has a water holding capacity of between 3 and 7 grams of water per gram of dry single cell protein product. For example, 5.57 g/g of dry pellet. The single cell protein product may have an oil holding capacity of about 1 to about 4 gram oil per gram of dried single cell protein product. For example, about 2.11 g/g of dry powder. The single cell protein product may have a brown/pink colour. The single cell protein product may have a structuring property with a viscoelastic Modulus of 2.59 Pa at the end of cooling.

Without being bound by theory, appearance and functionality may be altered based on dissolved oxygen content and methanol feed rate. For example, a lower methanol feeding rate and/or dissolved oxygen concentration may result in a single cell protein product having improved structuring properties.

As used herein structuring properties may be determined using methods such as rheumatological methods. For example, using a closed cavity rheometer device, such as the RPA elite model from TA Instruments, USA. A dried sample dispersion, is sealed in a container (to minimize moisture loss) and hydrated before testing. The prepared sample is placed within the CCR's closed cavity with disk geometry, under a constant pressure (e.g. 4 par) to prevent evaporation. The device conducts temperature sweeps (e.g., from 40° C. to 150° C.) at a specified rate (e.g., 10° C./min), maintaining a constant strain (e.g. 1%) and frequency (e.g. 1 Hz) within the linear viscoelastic region. After reaching a predefined temperature (e.g. 150° C.), the sample is cooled (e.g. to 50° C.) using dry air. The CCR records the storage modulus (G′) and loss modulus (G″), which are used to calculate the complex modulus (G*), indicative of the sample's viscoelastic behaviour and structural integrity post-cooling. Data analysis, performed by the rheometer's software, computes the complex shear modulus G* from G′ and G″, providing a quantitative measure of the sample dispersion's structural strength at the end of the cooling phase.

### Harvesting

The methods described herein may also include a step of harvesting or collecting the single cell protein product, for example collecting the cell mass produced by culturing of the methylotrophic microorganism (i.e. biomass), the culture or cell broth (culture media in which the microorganisms has been cultured), and/or the protein element of the culture broth and/or of the biomass. When the whole of the biomass is harvested, this may be referred to as a whole cell product.

The single cell protein product (biomass, culture broth and/or protein thereof) may be collected using any suitable method, such as one or more of flocculation, flotation, decanting, filtration and/or centrifugation, to separate the cell mass from the culture medium. The biomass may be centrifuged at 20,000 g. The biomass may be centrifuged for about 10 to 15 minutes. The biomass may be centrifuged for at least 10 minutes.

Flocculation refers the formation of a loose aggregation of discrete particles held together in a network-like structure by physical adsorption of macromolecules, bridging during chemical interaction (precipitation), or when the longer range van der Waals forces of attraction exceed the shorter range forces of attraction. Flotation refers to a phenomenon in which air is attached to the suspended phase dispersed in the dispersion medium to float to the limiting surface where the dispersion medium and air are in contact. It is a method of separating from water by stopping it on the surface of the water. Filtration refers to a process of separating solutes from a fluid, by passing the fluid through a filter medium across which certain solutes or suspensions cannot pass. Examples of filtration include ultrafiltration, membrane filtration, gravity and vacuum filtration.

Once separated the pelleted single cell protein product (biomass and/or protein thereof) may be extracted from the culture broth (supernatant). The cell culture broth may then be disposed of or kept for further processing as described herein.

The separated single cell protein product (biomass and/or protein thereof) may be washed after and/or before separation. For example, the pelleted biomass may be resuspended in a liquid such as demineralised water. The washed biomass may then be centrifuged to provide a washed biomass pellet. For example, centrifuged at 20,000 g. The washed biomass may be centrifuged for about 10 to 15 minutes. Alternatively or additionally, the biomass in culture medium may be washed by emersion in a wash liquid such as water. For example, by decanting off the culture broth or by diluting the culture broth with a wash liquid such as water.

The collected single cell protein product (biomass, culture broth and/or protein thereof) may be dried. For example, by freeze-drying (i.e. lyophilisation), rotary evaporation or spray drying. The single cell protein product (biomass, culture broth and/or protein thereof) may be frozen directly after separation or after washing as described herein. Methods of drying are well known in the art.

The separated biomass or washed biomass (single cell protein product) pellet may be frozen. The term “frozen” refers to freezing without lyophilising the separated biomass or washed biomass pellet. Without being bound by theory, freezing the separated biomass prior to carrying out any post-culture processing may provide a biomass that has improved functional properties in comparison to a biomass that has not been frozen. For example, freezing the separated biomass may provide a biomass with a fibrous structure. A fibrous structure may provide better functional and organoleptic properties of the biomass When used in or as food products such as meat analogues.

In some examples, harvesting may include any method that may disrupt or at least partially disrupt the cultured cells. Disruption of cells may refer to the lysis or partial lysis of the cells and includes any methods that may lead to the at least partial release of cellular components into the cell broth. For example, harvesting may include any one or more of sonification, heating, cooling, acidification, enzymatic treatment, chemically induced lysis and/or homogenisation. Disruption of cells may be carried out prior to separation. For example, harvesting may include homogenisation of the culture broth. For example, harvesting may include heating of the culture broth. For example, harvesting may include sonification of the culture broth. For example, harvesting may include acidification of the culture broth. For example, harvesting may include freezing of the culture broth. For example, harvesting may include addition of a chemical cell lysis compound to the culture broth. For example, harvesting may include lysis by addition of an enzyme to the culture broth. For example, one or more of a lysozyme, lysostaphin, zymolase, cellulose, protease or glycanase may be used to lyse cells.

In particular, when the method provides a microorganism or single cell protein product that has a relatively high concentration of Cytochrome C partial disruption of cultured cells in the culture broth as described herein may allow for a higher concentration of cytochrome C to be recovered in the final single cell protein product. For example, harvesting may include one or more of acidification as described herein, heating and/or homogenisation. As mentioned below, harvesting may be part of downstream processing.

### Post-Culture Processing (Downstream Processing)

The separated biomass (unwashed, washed, dried or frozen), cell broth and/or the protein thereof may be subjected to a number of different processing steps. These processing steps may each be used individually or in combination, for example, sequentially.

Post-culture processing may be carried out as part of harvesting or may be carried out after harvesting. The post-processing may include one or more of centrifugation, homogenisation, acidification, drying, filtration, heating, cooling, enzymatic treatment (e.g. removal of nucleic acids), rotary evaporation, gelation, texturizing, curdling, rehydrating and/or freezing.

Homogenisation refers to process of mixing one or more components, either similar or dissimilar, into a uniform mixture. Any conventional homogenizers can be used for the method disclosed herein. Some non-limiting examples of the homogenizer include stirring mixers, planetary stirring mixers, blenders and ultrasonicators.

Without being bound by theory, homogenisation may provide a single cell protein product (e.g. biomass or protein of the biomass and/or culture broth) that has increased viscosity in comparison to a single cell protein product (e.g. biomass or protein of the biomass and/or culture broth) that has not been homogenised. This may provide a biomass that has an increased yield of protein in the supernatant when a homogenised biomass is subsequently centrifuged. Homogenisation may also allow for the use of lower centrifuge speeds to separate a homogenised biomass pellet from its suspension liquid. In addition, homogenisation may also provide a single cell protein product (e.g. biomass or protein of the biomass and/or culture broth) with improved solubility, foaming and/or emulsifying properties.

Homogenization of the single cell protein product (e.g. biomass or protein of the biomass and/or culture broth) may also provide a stable dispersed emulsion. Such an emulsion may be useful in food products such as mayonnaise, salad dressings, and non-dairy creamers, and dairy analogue products such as yoghurt, cheese, ice cream, and milk alternatives. Without being bound by theory emulsifying may add essential fat ingredients to products.

After homogenisation, the homogenised single cell protein product (e.g. biomass or protein of the biomass and/or culture broth) may be subsequently centrifuged at a relatively low force and the pellet separated from the supernatant. For example, centrifuged at a force of less than 10,000 g. The supernatant may be processed (e.g. acidified) as described herein. For example, centrifuged at a force of equal to or less than 6500 g. For example, centrifuged at a force of equal to or less than 4000 g. For example, centrifuged at a force of 6500 g. For example, centrifuged at a force of 4000 g. For example, centrifuged at a force of 2000 g.

The biomass, protein or supernatant from any centrifugation step as described herein may be acidified. Acidification refers to a process of changing the alkalinity or neutrality of a system into acidity for example, by adding acid to the system. For example, the cultured biomass in culture medium prior to harvesting may be acidified by the addition of an acid, such as hydrochloric acid, other suitable acids will be known to those skilled in the art. The biomass may be acidified after harvesting. For example, the pellet produced by harvesting may be resuspended in a liquid and acidified. The supernatant obtained from harvesting or from centrifugation during any of the post-culture processes described herein may be acidified. Examples of methods of acidification can be found in EP0252900A2 which is incorporated herein by reference. In some examples, acidification, as described herein may include changing the pH of the target sample (e.g. biomass, culture broth, supernatant and/or protein of the biomass and/or culture broth) to a pH between 3 to 6. In some examples, to a pH of 4 to 5. For example, to a pH of 3, 4, 5, or 6. Acidification may be carried out in the same vessel as used to culture the microorganism or may be carried out in a second separate vessel to that used for the culturing.

Acidification may lead to precipitation of proteins from the biomass and/or supernatant (e.g. culture broth). Therefore, acidification may be used to form a protein concentrate. Acidification may also provide molecules that are not charged, thus aiding in flocculation of protein from the biomass or supernatant. Acidification may also decrease the amount of other constituents of the single cell protein product (e.g. biomass or protein of the biomass and/or culture broth) such as nucleic acids. Therefore, the use of acidification may provide a sample with increased concentration of protein.

Acidification may also provide for improved viscoelastic properties of the single cell protein product (e.g. biomass or protein of the biomass and/or culture broth).

After acidification, the acidified solution may be centrifuged in order to separate the protein concentrate from the suspension liquid. The pellet obtained from the low force centrifugation (for example, less than 10,000 g) may have properties suited for use in food products such as meat analogues and salad dressings. The pellet may have suitable OHC, WHC and rheological properties that are advantageous for use in food products such as meat analogues, salad dressings and the like. For example, an OHC of more than 1.5 grams of oil per gram of single cell protein product. For example, a viscosity of more than 300 mPa·s measured at 1S⁻¹. For example, centrifuged at a force of equal to or less than 6500 g. For example, centrifuged at a force of equal to or less than 4000 g. For example, centrifuged at a force of 6500 g. For example, centrifuged at a force of 4000 g. For example, centrifuged at a force of 2000 g.

The biomass may be resuspended after harvesting in a suspension liquid and centrifuged. For example, centrifuged at a relatively low force. For example, less than 10,000 g. For example, centrifuged at a force of equal to or less than 6500 g. For example, centrifuged at a force of equal to or less than 4000 g. For example, centrifuged at a force of 6500 g. For example, centrifuged at a force of 4000 g. For example, centrifuged at a force of 2000 g.

In some examples, the biomass may be centrifuged at a relatively high force. For example, at more than 10,0000 g. For example, at a force of equal to or more than 10,000 g. For example, at more than 15,000 g. For example, at a force of about 18,000 g. For example, when the cultivated biomass has a relatively high viscosity the cultivated biomass may be centrifuged at a relatively high speed. For example, a high viscosity may be equal to or more than 75 mPa·s. For example, a high viscosity may be equal to or more than 300 mPa·s. In some examples, when the cultivated biomass has a relatively high viscosity the cultivated biomass may be centrifuged at force of at least 2000 g. When the cultivated biomass has a relatively high viscosity the cultivated biomass may be centrifuged at a force of at least 4000 g. when the cultivated biomass has a relatively high viscosity the cultivated biomass may be centrifuged at a force of at least 6500 g.

The pellet from relatively high speed centrifugation may be used directly or may be further processed. The pellet obtained from high speed centrifugation may have properties suited for use in food products such as meat analogues and salad dressings. The pellet may have suitable OHC, WHC and rheological properties that are advantageous for use in food products such as meat analogues, salad dressings and the like.

The supernatant collected from the relatively high speed centrifugation may be acidified as described herein.

In some examples, after centrifugation, the pelleted single cell protein product (e.g. biomass or protein of the biomass and/or culture broth) may be subjected to additional post-culture processes. For example, drying by methods such as freeze-drying or toasting. After drying the biomass may be rehydrated. Without being bound by theory, a single cell protein product (e.g. biomass or protein of the biomass and/or culture broth) that has been centrifuged, freeze-dried and rehydrated may have a texture and appearance similar to meat and as such may be suited for use in a meat analogue.

In some examples, the biomass after, during or prior to harvesting may be subjected to cell lysis. “Cell lysis” refers to a process of releasing materials in a cell by disrupting the cell membrane. For example, cell lysis by physical methods such as sonication, ice shock or electroporation or chemical methods such as exposing the biomass to surfactants or enzymes that disrupt the cell membrane of the microorganism.

Without being bound by theory, cell lysis may provide a biomass that has improved solubility.

In some examples, the single cell protein product (e.g. biomass or protein of the biomass and/or culture broth) after, during or prior to harvesting may be enzymatically treated. For example, treated using enzymes that digest or degrade nucleic acids or degrade the cell membrane to cause cell lysis.

In some examples, single cell protein product (e.g. biomass or protein of the biomass and/or culture broth) after harvesting may be heated. Heating the biomass may denature proteins and alter the hydrophobicity of the biomass. This may increase the WHC and alter the properties of the biomass, such as rheological properties. Single cell protein product (e.g. biomass or protein of the biomass and/or culture broth) may be dried, for example, by freeze-drying, prior to being heated.

In some examples, the post processing may include rotary evaporation. Rotary evaporation refers to a method of increasing the evaporation area by rotating at a fixed speed, thereby accelerating evaporation.

In some examples, the processing may include filtration. For example, filtration as described herein. In some examples, processing may include filtration to provide a concentrated retentate.

In some examples, the concentrated retentate may be frozen. In some examples, the concentrated retentate may be dried. In some examples, the concentrated retentate may be frozen and dried. In some examples, the concentrated retentate may be dried using one or more of freeze-drying, spay-drying and/or other drying methods.

In some examples, the processing may include texturizing processes. The term texturization refers to the development of an unstructured protein product into a structured protein product. Particularly, the conversion of an unstructured protein product with no visible grain or texture into a structured protein product with a definite shape having the consistency of a desired food product. For example, mimetic of animal meat, a dairy product or poultry egg. Texturization methods and processes include acid gelation, extrusion processes and shear cell processes. These processes apply pressure and heat to the singe cell protein product to denature the protein and apply shear forces to form protein into defined structures such as fibres.

In some examples, the processing may also include gelation. Gelation refers to the process of forming a gel. A gel refers to a colloidal system in which a network of particles spans the volume of a liquid medium. Methods of gelation that may be used include acid gelation and/or heat gelation.

In some examples, the harvesting (or downstream processing) may be the same. For example, harvesting may comprise centrifuging at a force of at least 2000 g. In some examples, centrifuging at a force of at least 10,000 g. In some examples, centrifuged at a force equal to or less than 6500 g. For example, centrifuged at a force of equal to or less than 4000 g. For example, centrifuged at a force of 6500 g. For example, centrifuged at a force of 4000 g. For example, centrifuged at a force of 2000 g. For example, centrifuged at a force of 18000 g. For example, centrifuged at a force of 20000 g.

In some examples, after harvesting (e.g. by acidification, centrifugation and/or flocculation) the harvested single cell protein product (e.g. the culture broth (including disrupted cells and cell culture medium), the separated biomass, the cell culture medium and/or protein thereof) may be subjected to filtration. In some examples, after harvesting (e.g. by acidification, centrifugation and/or flocculation) the harvested single cell protein product may be subjected to drying. For example, freeze drying or spray drying.

In some examples, harvesting and downstream processing may include the steps of centrifuging to obtain a pellet and drying the pellet. For example, the harvesting and processing may include, centrifuging the cultured microorganism and culture media (i.e. culture broth) at a force of at least 2,000 g. In some examples, a force of 18,000 g. In some examples, a force of 20,000 g. The pellet may then be frozen, for example, frozen for at least 5 hours. In some examples, frozen for about 24 hours. The frozen pellet may then be dried, for example, freeze dried. For example freeze dried till a dry matter content of 95% is achieved. The freeze dried pellet may then ground to a powder. The single cell protein product produced using such processing methods may have a protein content of at least 60%. For example, about 88%. The single cell protein product produced using such processing methods may have water holding capacity of between 2 and 10 grams of water per gram of dry single cell protein product. For example, about 8.5 g/g of dry pellet. The single cell protein product produced using such processing methods may have a solubility of at least 40%. For example, about 50.9%. The single cell protein product produced using such processing methods may have an oil holding capacity of between 1 and 4 gram oil per gram of dried single cell protein product. For example, about 2.2 gram oil per gram of dried single cell protein product.

In some examples, harvesting and downstream processing may include the steps of centrifuging to obtain a pellet and spray drying. For example, the harvesting and processing may include, centrifuging the cultured microorganism and culture media (e.g. culture broth) at a force of at least 2000 g. In some examples, a force of 18,000 g. In some examples, a force of 20,000 g. The pellet may then be frozen, for example, frozen for at least 5 hours. In some examples, frozen for about 24 hours. In some examples, the pellet is not frozen. After centrifugation or freezing, the pellet may then be spray dried. The single cell protein product produced using such processing methods may have a protein content of at least 60%. For example, about 85%. The single cell protein product produced using such processing methods may have water holding capacity of between 2 and 6 grams of water per gram of dry single cell protein product. For example, about 4 g/g of dry pellet. The single cell protein product produced using such processing methods may have a solubility of at least 10%. For example, about 15.1%. The single cell protein product produced using such processing methods may have an oil holding capacity of between 0 and 4 gram oil per gram of dried single cell protein product. The single cell protein product produced using such processing methods may have a structuring property with a viscoelastic Modulus of at least 60 Pa at the end of cooling. For example, about 78.9 Pa. The single cell protein product produced using such processing methods may have be suited for use in food products such as meat, dairy, fish or egg analogues.

In some examples, harvesting and downstream processing may include the steps of acidifying, centrifuging to obtain a pellet and drying. For example, the harvesting and processing may include, acidifying the cultured microorganism and cell culture media (e.g. culture broth). For example, the pH may be brought to a value of pH 4.5. For example, by the addition of an acid such a phosphoric acid. The acidified cultured microorganism and cell culture media may then be centrifuged at a force of at least 2,000 g. In some examples, a force of 6,500 g. In some examples, a force of 4,000 g. In some examples, a force of 2,000 g. The pellet may then be frozen, for example, frozen for at least 5 hours. In some examples, frozen for about 24 hours. The frozen pellet may then be freeze dried. For example freeze dried till a dry matter content of 95% is achieved. The freeze dried pellet may then ground to a powder. The single cell protein product produced using such processing methods may have a protein content of at least 60%. For example, about 82%. The single cell protein product produced using such processing methods may have water holding capacity of between 2 and 6 grams of water per gram of dry single cell protein product. For example, about 2.8 g/g of dry pellet. The single cell protein product produced using such processing methods may have a solubility of at least 10%. For example, about 25.2%. The single cell protein product produced using such processing methods may have an oil holding capacity of between 0 and 4 gram oil per gram of dried single cell protein product. For example, about 1.6 gram oil per gram of dried single cell protein product. The single cell protein product produced using such processing methods may have a structuring property with a viscoelastic Modulus of at least 25 Pa at the end of cooling. For example, about 32 Pa.

In some examples, harvesting and downstream processing may include the steps of acidifying, centrifuging to obtain a pellet and drying. For example, the harvesting and processing may include, acidifying the cultured microorganism and culture media (culture broth). For example, the pH may be brought to a value of pH 4.5. For example, by the addition of an acid such a phosphoric acid. The culture broth may then be centrifuged at a force of at least 2,000 g. In some examples, a force of 6,500 g. In some examples, a force of 4,000 g. In some examples, a force of 2,000 g. The pellet may then be neutralised. For example, by bringing the pH to a value of about pH 6.5. For example, by the addition of a base or alkaline. For example, by adding ammonium hydroxide to the pellet. The neutralised pellet may then be frozen, for example, frozen for at least 5 hours. In some examples, frozen for about 24 hours. The frozen pellet may then be freeze dried. For example freeze dried till a dry matter content of 95% is achieved. The freeze dried pellet may then ground to a powder. The single cell protein product produced using such processing methods may have a protein content of at least 60%. For example, about 81%. The single cell protein product produced using such processing methods may have water holding capacity of between 2 and 6 grams of water per gram of dry single cell protein product. For example, about 5.3 g/g of dry pellet. The single cell protein product produced using such processing methods may have a solubility of at least 10%. For example, about 20.2%. The single cell protein product produced using such processing methods may have an oil holding capacity of between 0 and 4 gram oil per gram of dried single cell protein product. For example, about 2 gram oil per gram of dried single cell protein product. The single cell protein product produced using such processing methods may have a structuring property with a viscoelastic Modulus of at least 25 Pa. For example, about 30.6 Pa.

In some examples, harvesting and downstream processing may include the steps of flocculating, centrifuging to obtain a pellet and drying. For example, the harvesting and processing may include, flocculating the cultured biomass and/or culture media (culture broth). For example, using a flocculating agent such as CaCl₂). After flocculating, the flocculated pellet may be centrifuged at a force of at least 2,000 g. In some examples, a force of 6,500 g. In some examples, a force of 4,000 g. In some examples, a force of 2,000 g. The pellet may then be frozen, for example, frozen for at least 5 hours. In some examples, frozen for about 24 hours. The frozen pellet may then be freeze dried. For example freeze dried till a dry matter content of 95% is achieved. The freeze dried pellet may then ground to a powder. The single cell protein product produced using such processing methods may have a protein content of at least 60%. For example, about 82%. The single cell protein product produced using such processing methods may have water holding capacity of at least 4 grams of water per gram of dry single cell protein product. For example, about 6 g/g of dry pellet. The single cell protein product produced using such processing methods may have a solubility of at least 10%. For example, about 32.7%. The single cell protein product produced using such processing methods may have an oil holding capacity of between 0 and 3 g gram oil per gram of dried single cell protein product. For example, about 1.9 gram oil per gram of dried single cell protein product. The single cell protein product produced using such processing methods may have a structuring property with a viscoelastic Modulus of at least 25 Pa at the end of cooling. For example, about 28.9 Pa.

In some examples, harvesting and downstream processing may include the steps disrupting the culture cells, acidifying, centrifuging to obtain a supernatant, filtering the supernatant to obtain a retentate and drying. For example, the harvesting and processing may include, disrupting the cell of the cultured microorganism. The disrupted cells and culture media (culture broth including disrupted cells), may then be acidified. For example, the pH may be brought to a value of pH 3.5. For example, by the addition of an acid such a phosphoric acid. The acidified culture broth may then be centrifuged at a force of at least 2,000 g. In some examples, a force of 4,000 g. In some examples, a force of 6,000 g. In some examples, a force of 18,000 g. In some examples, a force of 20,000 g. The supernatant may then be filtered. In some example, the pellet may be discarded or stored. In some examples, the pellet is processed as described herein. The supernatant may be filtered using any suitable method. In some example, filtration comprises filtering the supernatant to remove any particulates greater than 10 kDa. For example, using a 10 kDa membrane. In some examples, filtration comprises ultrafiltration. As used herein, “ultrafiltration” refers to the filtration of liquid through a membrane having a pore size of about 0.001 μm to about 0.02 pm. Filtration provides a retentate. In some examples, the retentate may be concentrated. That is to say that the concentration of the constituent components of the retentate may be present at a higher concentration that before filtration. The retentate may then be frozen. For example, frozen for about 24 hours. In some examples, the retentate is not frozen. In some example, the frozen retentate or retentate may be dried. For example, spray dried or freeze dried as described herein. This harvesting and processing may be particularly suited for processing of microorganisms cultured to provide a relatively high concentration of cytochrome C.

For example, the supernatant and/or concentrated retentate may have a cytochrome C concentration of at least 10×10 mM. For example, the supernatant and/or concentrated retentate may have a cytochrome C concentration of about 15 ×10⁻³ mM. For example, the supernatant and/or concentrated retentate may have a cytochrome C concentration of about 17 ×10⁻³ mM. For example, the supernatant and/or concentrated retentate may have a cytochrome C concentration of about 20 ×10⁻³ mM. For example, the supernatant and/or concentrated retentate may have a cytochrome C concentration of about 25 ×10⁻³ mM.

### Properties

The methods provided herein provide single cell protein products that may have novel and advantageous properties. These properties may be imparted or controlled by the culturing method used to culture the microorganism or by the post-culture processing methods used during and/or after harvesting.

For example, single cell protein products produced by a method of restricted cultivation have different rheological properties when compared to a single cell protein product (e.g. biomass or protein of the biomass and/or culture broth) produced by a unrestricted cultivation method. For example, the single cell protein product (e.g. biomass or protein of the biomass and/or culture broth) produced by a method of restricted cultivation may have different viscoelastic properties in comparison to single cell protein product (e.g. biomass or protein of the biomass and/or culture broth) produced by an unrestricted cultivation method. The term “viscoelastic” refers to viscous fluids having elastic properties, i.e., the liquid at least partially returns to its original form when an applied stress is released.

In some examples, the single cell protein product (e.g. biomass or protein of the biomass and/or culture broth) may have a viscoelastic modulus in the range of 1-10 kPa when a 40% dry matter sample is heated up from 40 to 150° C., and in the range of 5-15 kPa when subsequently cooled down from 150-50° C.

Single cell protein products (e.g. biomass or protein of the biomass and/or culture broth) produced by a unrestricted cultivation method may have functional properties that are similar to plant based proteins such as pea protein isolate, pea protein concentrate, soy protein isolate and/or soy protein concentrate.

Single cell protein product (e.g. biomass or protein of the biomass and/or culture broth) produced by an unrestricted cultivation method may have a relatively low viscosity. For example, a low viscosity may be equal to or less than 2000 mPa·s measured at 1S⁻¹. For example, a low viscosity may be equal to or less than 50 mPa·s measured at 1S⁻¹.

Single cell protein product (e.g. biomass or protein of the biomass and/or culture broth) produced by a restricted cultivation method may have a relatively high viscosity. For example, a high viscosity may be equal to or more than 75 mPa·s measured at 1S⁻¹. For example, a high viscosity may be equal to or more than 300 mPa·s measured at 1S⁻¹.

Single cell protein product (e.g. biomass or protein of the biomass and/or culture broth) produced by an unrestricted or batch cultivation method may have a protein content of at least 45% determined by the Dumas method with a Nitrogen Conversion Factor of 4.88. For example, at least 45%, 46%, 47%, 48%, 49%, 50%, 51%, 52%, 53%, 54%, 55%, 60%, 65%, 70%, 75%, or 80%.

Single cell protein product (e.g. biomass or protein of the biomass and/or culture broth) produced by a restricted or fed-batch cultivation method may have a protein content of at least 60% determined by the Dumas method with a Nitrogen Conversion Factor of 4.88. For example, at least 60%, 65%, 70%, 75%, or 80%.

Protein content of biomasses can be determined using any suitable methods known in the art. For example, by using the Dumas method which calculates the nitrogen content to determine protein content. The Dumas method uses a nitrogen conversion factor which is based on the amino acid profile. The nitrogen conversion factor may be determined experimentally or may be derived from a related compound. For example, the nitrogen conversion factor used may be that for biomass product such as Pruteen which is 4.88 (i.e. Protein %=Nitrogen % ×4.88). Other methods that may be used include the Biuret Method, Lowery assay, BCA assay, Bradford assay, or the Kjeldahl method as well as fluorescent based methods.

Single cell protein product (e.g. biomass or protein of the biomass and/or culture broth) produced by an unrestricted or batch cultivation method may have a water holding capacity (WHC) of at least 2.5 gram water per gram of dry single cell protein product. For example, at least, 2.5, 3, 3.5, 4, 4.5, 5, 5.5, or 6 gram water per gram of dry single cell protein product. For example, about 6.2 gram water per gram of dry single cell protein product.

Single cell protein product (e.g. biomass or protein of the biomass and/or culture broth) produced by a restricted or fed-batch cultivation method may have a water holding capacity (WHC) of at least 6 gram water per gram of dry single cell protein product. For example, at least, 6, 7, 8, 9 or 10 gram water per gram of dry single cell protein product. For example, about 6.6 g/g dry single cell protein product.

Water-holding capacity (WHC) (or water-binding capacity, or water-absorption capacity) is a measure of the total amount of water that can be absorbed per unit weight of a biomass (pelleted or powdered). This property is based on the direct interaction of protein molecules with water and other solutes. Any suitable method known in the art may be used to measure WHC. For example, a known amount of dry sample (e.g. biomass) may be first hydrated. The sample may then centrifuged. The pellet may then be weighed and dried. The WHC is calculated from the weight of the wet sample (pellet) relative to the dried sample. In some examples, WHC maybe determined by a method comprising the steps of dissolving dried single cell protein product (e.g. biomass or protein of the biomass and/or culture broth) in a liquid (e.g. water) to a known weight to volume concentration. Then centrifuging the sample. For example at 20,000G. The supernatant is then discarded and the wet pellet is weighed. The sample is then dried, for example, overnight and the dried pellet is then weighed.

The WHC is calculated as the weight difference between the wet pellet and dry pellet divided by the weight of the dried pellet, with a unit of g water/g dry pellet.

In some examples, single cell protein product may have an WHC of between 2 and 10 gram water per gram of dry single cell protein product. For example, about 8.5 gram water per gram of dry single cell protein product. For example, about 4 gram water per gram of dry single cell protein product. For example, about 2.8 gram water per gram of dry single cell protein product. For example, about 5.3 gram water per gram of dry single cell protein product. For example, about 5.3 gram water per gram of dry single cell protein product. For example, about 6 gram water per gram of dry single cell protein product. For example, about 5.97 gram water per gram of dry single cell protein product. For example, about 4.7 gram water per gram of dry single cell protein product. For example, about 3.29 gram water per gram of dry single cell protein product. For example, about 5.57 gram water per gram of dry single cell protein product.

Single cell protein product (e.g. biomass or protein of the biomass and/or culture broth) produced by an unrestricted or batch cultivation method may have an oil holding capacity (OHC) of at least 1 gram oil per gram of dried single cell protein product. For example, about 2.5 g oil/g dry single cell protein product.

Single cell protein product (e.g. biomass or protein of the biomass and/or culture broth) produced by a restricted or fed-batch cultivation method may have an oil holding capacity (OHC) of at least 1.5 gram oil per gram of dry single cell protein product. For example, about 2.4 g oil/g dry single cell protein product.

In comparison to plant based proteins, the single cell protein products as described herein may have increased OHC. This increased OHC may provide advantages in relation to the binding of oils (such as fat analogues) and other agents such as flavouring agents in comparison to plant proteins. Therefore, the biomasses and protein products derived therefrom as described herein with a higher OHC in comparison to plant proteins may be suited for use in particular foods such as meat analogues.

OHC refers to the amount of oil that a sample can absorb per unit of weight. An ingredient with high OHC can be used to improve the organoleptic properties of a food product (for example, texture, mouthfeel and flavour) of a food product, such as dairy product or meat analogue. Any suitable method known in the art may be used to measure OHC. For example, a known amount of dry sample may be dispersed in an oil. The sample may then be centrifuged. The pellet is then weighed. The OHC is calculated from the weight of the wet sample (pellet) relative to the initial dry sample.

In some examples, single cell protein product may have an OHC of between 1 and 4 g oil/g dry single cell protein product. For example, about 1.44 g oil/g dry single cell protein product. For example, about 1.73 g oil/g dry single cell protein product. For example, about 1.66 g oil/g dry single cell protein product. For example, about 2.11 g oil/g dry single cell protein product. For example, about 2.2 g oil/g dry single cell protein product. For example, about 1.6 g oil/g dry single cell protein product. For example, about 2 g oil/g dry single cell protein product. For example, about 1.9 g oil/g dry single cell protein product.

Single cell protein product (e.g. biomass or protein of the biomass and/or culture broth) produced by an unrestricted or batch cultivation method may have solubility of at least 20%. For example, at least 20, 25, 30, 40 or 50%. For example, 36%.

Single cell protein product (e.g. biomass or protein of the biomass and/or culture broth) produced by a restricted or fed-batch cultivation method may have solubility of at least 20%. For example, about 20.4%.

In some examples, single cell protein product may have a solubility of at least 15%. For example, a solubility of about 50%. For example, a solubility of about 15%. For example, a solubility of about 25%. For example, a solubility of about 20%. For example, a solubility of about 33%.

Single cell protein product (e.g. biomass or protein of the biomass and/or culture broth) produced by an unrestricted or batch cultivation method may have a viscosity of less than 200 mPa·s measured at 1S⁻¹. In some example, less than 75 mPa·s measured at 1S⁻¹.

Single cell protein product (e.g. biomass or protein of the biomass and/or culture broth) produced by a restricted or fed-batch cultivation method may have a viscosity of more than 75 mPa·s measured at 1S⁻¹. In some example, more than 300 mPa·s measured at 1S⁻¹.

Viscosity refers to a measure of the degree to which a fluid or aqueous composition, such as a hydrocolloid, resists forces that tend to cause it to flow. Various viscosity units that may be used herein include centipoise (cPs) and pascal seconds (Pa· s). One centipoise is one percent of one poise; one poise is equal to 0.100 kg.m−1·s−1. Examples of methods of measuring viscosity include, but are not limited to, a glass capillary viscometer or a vibrating needle viscometer, a rheometer, a rotational rheometer, and the inclined plane test, but any suitable method can be utilized. In some examples, dried single cell protein product is dispersed at a known weight to volume concentration in a liquid such as water in order to determine viscosity. In some examples, viscosity is determined an Anton Paar (TA instrument, The Netherlands) with a flow sweep applied to the dispersed single cell protein product at 20° C. from 0.1 to 100 s−1, and the result is obtained from the device with the unit of mPa/s.

The structuring properties of the single cell protein product are defined by its viscoelastic modulus, measured in Pa at the conclusion of a specified cooling phase within the testing protocol. Utilizing the Closed Cavity Rheometer (CCR), as detailed herein, the viscoelastic modulus of the single cell protein product is determined post-cooling, which provides a quantitative measure of its structural integrity and performance capabilities under prescribed conditions. The viscoelastic modulus serves as a critical parameter in assessing the functional and mechanical properties of the single cell protein product. It directly relates to the product's ability to maintain its form and structural coherence under mechanical stress and strain, which may affect both processing and end-use applications.

In some examples, the single cell protein product may a have a structuring property value of at least 2 Pa. In some examples, the single cell protein product may a have a structuring property value of from 2 Pa to 90 Pa. For example, the single cell protein product may a have a structuring property value of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 Pa. In some examples, the single cell protein product may a have a structuring property value of 10 to 90 Pa. For example, the single cell protein product may a have a structuring property value of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 Pa. In some examples, the single cell protein product may a have a structuring property value of 10 to 80 Pa. For example, the single cell protein product may a have a structuring property value of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 Pa. In some examples, the single cell protein product may a have a structuring property value of 25 to 80 Pa. For example, the single cell protein product may a have a structuring property value of 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 Pa. In some examples, the single cell protein product may a have a structuring property value of 15 to 25. For example, the single cell protein product may a have a structuring property value of 15, 16, 17, 18, 19, 20, 21, 22, 23, 24, or 25 Pa. In some examples, the single cell protein product may a have a structuring property value of 25 to 35 Pa. For example, the single cell protein product may a have a structuring property value of 25, 26, 27, 28, 29, 30, 31, 32, 33, 34, or 35 Pa. In some examples, the single cell protein product may a have a structuring property value of 35 to 45 Pa. For example, the single cell protein product may a have a structuring property value of 35, 36, 37, 38, 39, 40, 41, 42, 43, 44, or 45 Pa.

In some examples, the single cell protein product may a have a structuring property value of about 41 Pa. In some examples, the single cell protein product may a have a structuring property value of about 15 Pa. In some examples, the single cell protein product may a have a structuring property value of about 18 Pa. In some examples, the single cell protein product may a have a structuring property value of about 2.5 Pa. In some examples, the single cell protein product may a have a structuring property value of about 78 Pa. In some examples, the single cell protein product may a have a structuring property value of 32 Pa. In some examples, the single cell protein product may a have a structuring property value of 30 Pa. In some examples, the single cell protein product may a have a structuring property value of 28 Pa.

The structuring property value as described herein may be measured by using a 40% dry matter sample heated up from 40 to 150° C. at a rate of 10° C./min maintaining a 1% constant strain and 1 Hz frequency within the linear viscoelastic region and then subsequently cooled down from 150 to 50° C. The storage modulus (G′) and loss modulus (G″), are then used to calculate the complex modulus (G*), indicative of the sample's viscoelastic behaviour and structural integrity post-cooling.

Other properties such as appearance (e.g. colour) and texture may also be imparted on the cultivation method used.

Properties such as gelation, viscosity, emulsification, and/or foamability may differ between single cell protein product (e.g. biomass or protein of the biomass and/or culture broth) produced using different cultivation methods.

Properties such as gelation, viscosity, emulsification, and/or foamability may differ between single cell protein product (e.g. biomass or protein of the biomass and/or culture broth) produced using cultivation methods as described herein may be improved in comparison to plant proteins such as pea protein isolate, pea protein concentrate, soy protein isolate and/or soy protein concentrate.

In some examples, the methods described herein may provide for a relatively high level of production of Cytochrome C by the microorganism. As such, the methods described herein may provide a single cell protein product (such as the culture broth which includes cultured cells as described herein and culture medium optionally harvested and/or processed as described herein) that has a concentration of Cytochrome C of at least 10×10⁻³ mM. For example, at least or about 10, 11, 12, 13, 14, 15, 16, 17, 18, 19, 20, 21, 22, 23, 24, 25, 27, 28 29, or 30 ×10⁻³ mM of cytochrome C.

In some examples, the methods described herein may provide a single cell protein product (such as a product derived from separated and optionally processed biomass) that has a concentration of Cytochrome C of at least a 200 μmol/mg of dry weight. For example, at least 200, 250, 300, 350, 400, 500, 500, 700, 800, 900, or 1000 μmol/mg of dry weight of the single cell protein product.

In some examples, when the single cell protein product includes a relatively high concentration of Cytochrome C, the single cell protein product may have a red, pink, beige, off-white or reddish-brown colour.

The term “gelation” refers to the process of becoming gel, e.g., transition from a viscous liquid state to a gel state. As such, the single cell protein product (e.g. biomass or protein of the biomass and/or culture broth) as described herein may have improved gelling properties in comparison to plant proteins.

“Emulsification” refers to any process of producing an emulsion. As such, the single cell protein product (e.g. biomass or protein of the biomass and/or culture broth) as described herein may have improved emulsion forming and/or stabilising properties in comparison to plant proteins.

“Foamability” refers to the ability of a substance (such as a protein) to rapidly adsorb at the air-liquid interface during whipping or bubbling, and form a viscous viscoelastic film via intermolecular interactions. As such, the single cell protein product (e.g. biomass or protein of the biomass and/or culture broth) as described herein may have improved foam forming and/or stabilising properties in comparison to plant proteins.

### Methanol Source

The methods described herein include methanol as a metabolite for the microorganism cultured.

In some examples, the methanol is obtained using renewable or environmentally friendly methods (referred to as sustainable methanol). For example, the methanol may be “e-methanol”. E-methanol is methanol produced using renewable energy sources. For example, the methanol may be obtained from recycled carbon dioxide, biogas, biomass, waste, or sewage sludge.

In some examples, e-methanol may be produced by chemically combining CO₂ and hydrogen. First, hydrogen is produced in an electrolyzer. The hydrogen is converted into methanol in a reactor by catalysis with carbon dioxide (CO₂). To produce ‘green’ (i.e. carbon-neutral) e-Methanol, hydrogen from renewable electricity is used in combination with biogenic CO₂. The CO₂ used can be obtained via carbon-capture processes, for example from industrial waste gases (biogenic or non-biogenic CO₂), or captured directly from the air (“Direct Air Capture” or “DAC”). This process avoids the emission of further, climate-damaging carbon dioxide. In addition, methanol synthesis produces water and waste heat, which in turn can be used within a closed-loop system. The water is fed back directly into the electrolysis process, where it serves as feedstock for further hydrogen production.

The use of e-methanol allows for a decoupling of food production from the use or arable or pastural land and is therefore less land intensive and better for the environment.

Even though 40-50% of the electricity is lost when converting the electricity to methanol, this is offset by efficiency of the microorganisms as described herein. In the fermentation process around 45% of the methanol is turned into biomass. This provides a total efficiency of about 4.5% versus the ~0.01-0.1% when using photosynthesis (arable farming) to create the edible plant parts for food. Therefore, the use of e-methanol as a feedstock for the microrganisms described herein provides a 540 fold increase in efficiency in comparison to sunlight-to-beef.

Other sustainable methanol sources include, bio-methanol, grey methanol, brown methanol and blue methanol. “Biomethanol” (also referred to as green methanol) refers to a type of methanol that is produced by gasification of organic material (such as biomass, or solid waste) to synthesis gas (also known as syngas), followed by methanol synthesis. Blue methanol is produced using blue hydrogen in combination with carbon capture technology, vastly reducing well-to-tank carbon dioxide emissions. Grey methanol is produced from natural gas and may not significantly reduce well-to-wake carbon dioxide emissions. Brown methanol is produced from coal.

In some examples, the methanol may be a mixture of conventionally produced methanol and any sustainable methanol as described herein.

### Food Products

The single cell protein products (e.g. biomass, culture broth and/or protein thereof) produced by methods described herein may be used in a number of different food products. The single cell protein products (e.g. biomass, culture broth and/or protein thereof) described herein are suitable for use in food products. Food product refers to any edible, comestible and/or ingestible product for example, food or drink.

Food products include meat (e.g. pork, beef, lamb, kangaroo, goat and horse), poultry (e.g. turkey, chicken, duck, guinea fowl, geese, quail, pigeon, ostrich and pheasant), or seafood (e.g. bony fishes, crustaceans, molluscs, cephalopod molluscs, and echinoderms). Food products also include confectionery and bakery products (including dough, bread, biscuits, crackers, muffins, pastries, cakes, pies, custard pies and cookies), ice cream (including pulsed ice cream, popsicles, frozen yogurt, ice cream, sorbet, sorbet and soy, oats, legumes and rice ice cream), dairy products (including drinking milk, cheese, yogurt and sour-milk drinks), cheeses (including natural cheeses and processed cheeses), butter, margarine, sweet and savoury snacks (including fruit snacks, chips/crisps, tortilla/corn chips, popcorn, pretzels, sweets and nuts), hot and cold drinks (including drink mixes, concentrates, juices, carbonated drinks, still drinks, alcoholic drinks, and soft drinks).

Therefore, single cell protein products (e.g. biomass, culture broth and/or protein thereof) described herein may be used to supplement or replace the naturally occurring ingredients such as protein and fats. This may allow for more food products to be produced that have improved nutritional properties and also do not require traditionally farmed ingredients.

The single cell protein products (e.g. biomass, culture broth and/or protein thereof) may be used as a foaming agent. As used herein, the term “foam” refers to a two-phase system in which gas cells are enclosed by liquid.

Single cell protein products (e.g. biomass, culture broth and/or protein thereof) that have been post-culture processed by homogenisation may have properties suited to use as a foaming agent.

For example, the single cell protein products (e.g. biomass, culture broth and/or protein thereof) may be used in any food product that comprises a foam. For example, whipped products such as whipped cream. For example, in a beverage that includes a foam such as carbonated drinks

The single cell protein products (e.g. biomass, culture broth and/or protein thereof) may be used as an emulsifying agent or emulsifier. That is to say that the single cell protein products (e.g. biomass, culture broth and/or protein thereof) may be used to stabilise an emulsion. For example, the single cell protein products (e.g. biomass, culture broth and/or protein thereof) may be used to stabilise a food product that includes an emulsion. Emulsion refers to a heterogeneous preparation composed of two immiscible liquids [e.g. lipophilic (oily) phase and aqueous or hydrophilic phase] where one immiscible phase is dispersed in the other uniformly, for example in the form of drops. The drops may be stabilised by an emulsifying agent (emulsifier) as described herein. The emulsion may be oil-in-water emulsion. The term oil-in-water emulsion refers to a stable emulsion in which the aqueous (water) phase is the dispersion medium and the oil component is the dispersed phase.

Examples of foods that may include the single cell protein products (e.g. biomass, culture broth and/or protein thereof) as an emulsifier include mayonnaise, margarine, meats, salad dressings, chocolate, peanut butter and other nut butters, shelf-stable frostings, cookies, crackers, creamy sauces, breads, baked products and ice cream.

Single cell protein products (e.g. biomass, culture broth and/or protein thereof) that has been post-culture processed by homogenisation may have properties suited to use as an emulsifier.

The biomass as a whole or protein products derived therefrom (single cell protein products as described herein)) may be used as an egg analogue. An “egg analogue” as used herein is a substitute of egg in the major characteristics of purpose and usage. For example, in cooking, eggs are frequently used as an emulsifier and as a thickener; in baking, eggs can add structure and leavening to cakes and cookies; in other applications, such as in drinks (such as cocktails) egg whites may be used as a foaming agent.

Egg analogues normally include a protein element along with other components such as fats and polysaccharides. The single cell protein products (e.g. biomass, culture broth and/or protein thereof) may be used as the protein element of an egg analogue. For example, the single cell protein products (e.g. biomass, culture broth and/or protein thereof) described herein may be used as an alternative to vegetable protein used in egg analogues as described in WO2022234108A1 and WO2023279189A1 which are incorporated herein by reference.

The single cell protein products (e.g. biomass, culture broth and/or protein thereof) may be used as a substituent for egg in an egg containing food product. For example, as a substituent for egg in sauces such as mayonnaise or as a substituent for egg in salad dressings.

The single cell protein products (e.g. biomass, culture broth and/or protein thereof) as described herein may be used as a dairy analogue or as part of a dairy analogue.

As used herein, the term “dairy analogue” refers to a non-dairy or vegan product having a similar flavour and texture to that of a conventional dairy product. The single cell protein products (e.g. biomass, culture broth and/or protein thereof) may be used as a substituent for a dairy product such as cream or milk in a dairy containing food product. For example, as a substituent for milk in in salad dressings, cream and cream containing products, baked goods, cheese, ice cream and the like. “Dairy product” refers to a food product or other product obtained by the processing of milk. As such, the single cell protein products (e.g. biomass, culture broth and/or protein thereof) as described herein may be for use as an analogue in any product that would normally include milk obtained from an animal.

The dairy product may be an analogue of a cheese i.e. an analogue of a cheese formed by coagulation of the milk protein casein.

The dairy product may be an analogue of an acidic dairy product i.e. yogurt and acid cheeses, during the production of which acid whey is formed as by-product, such as cottage cheeses, strained yogurts or Greek-style yogurts.

Single cell protein products (e.g. biomass, culture broth and/or protein thereof) that has been cultured by an unrestricted fermentation as described herein may be suited for use as a dairy analogue due to its properties such as colour, rheology, viscosity, and solubility. In addition, single cell protein products (e.g. biomass, culture broth and/or protein thereof) that have been post-culture processed by homogenisation may also have properties suited to use as a dairy analogue.

The single cell protein products (e.g. biomass, culture broth and/or protein thereof) described herein may be used in the making of meat analogues.

Single cell protein products (e.g. biomass, culture broth and/or protein thereof) that have been cultured by unrestricted and restricted fermentation or restricted fermentation as described herein may be suited for use as a meat analogue due to its properties such as appearance, texture, structure, rheology, viscosity, OHC, WHC and solubility.

In some examples, single cell protein products (e.g. biomass, culture broth and/or protein thereof) that has been post-culture processed by homogenisation may also have properties suited to use as or in a meat analogue.

In some examples, single cell protein products (e.g. biomass, culture broth and/or protein thereof) that has been post-culture processed by centrifugation, freeze-drying and rehydration may have properties suited to use as or in a meat analogue

A meat analogue refers to a food product that is not produced by the slaughter of an animal but has structure, texture, aesthetic qualities, and/or other properties comparable or similar to those of slaughtered animal meat, such as livestock (e.g., beef, pork), game (e.g., venison), poultry (e.g., chicken, turkey, duck), and/or fish or seafood. The term refers to uncooked, cooking, and cooked meat-like food products. Seafood refers to the marine and freshwater species in Phylum Arthropoda, Class Malacostraca, Orders Decapoda and Euphausiacea (e.g. shrimp, crayfish, lobsters and crabs); Phylum Mollusca, Classes Bivalvia, Gastropoda and Cephalopoda (e.g. shellfish) Phylum Echinodermata, Classes Echinoidea and Holothuroidea (e.g. sea urchins and sea cucumbers); Phylum Chordata, Class Actinopterygii, Orders Pleuronectiformes, Perciformes, Scorpaeniformes, Gadiformes, Anguilliformes (e.g. pelagic fish, demersal fish and reef fish).

The meat analogue may be a whole cut of meat. For example, a steak, chop, shoulder, belly or rib cut. The meat analogue may be a processed meat analogue. Processed meat analogues refer to meat analogues that mimic meat products that have undergone some form of processing, such as mincing or grinding. For example, the meat analogue may be minced meat analogue. The meat analogue may be a sausage or similar form of food product.

The single cell protein products (e.g. biomass, culture broth and/or protein thereof) described herein may be used as the protein element of a meat analogue.

The meat analogue may be made from a base material that includes a non-animal derived protein; the non-animal derived protein may comprise protein derived from the biomass described herein or include the biomass as a whole. Such meat analogues may additionally include a plant protein, such as a vegetable protein. The non-animal derived protein may additionally comprise a fungal protein, a protein extracted from a microorganism, or a recombinantly produced protein, for example, a microbially produced recombinant protein such as beef myoglobin. The non-animal derived protein may be in pure form of protein isolate, or a protein concentrate. The protein may be a defatted meal with a high protein content providing a protein content of greater than about 55%.

Meat analogues as used herein also refers to meat analogues formed from cultured cells. Cultured cell based meat analogues may also be known as cultured meat, in vitro meat, cellular agriculture products, or artificial meat. Such products are formed by in vitro culturing of non-human animal cells (for example, non-human animal myocytes) to form a structure that resembles cuts of meat obtained from a farmed animal. For example, see “Ng, Ee Theng, et al. “Cultured meat-a patentometric analysis.” Critical Reviews in Food Science and Nutrition (2021): 1-11.” And the references included therein.

The meat analogue may include one or more flavouring agents, colouring agents, flavour precursors, preservatives, spices, Maillard reaction precursors, seasonings or any combinations thereof.

A “flavouring agent” refers to a compound or salt or solvate thereof, that can be acceptably ingested, that induces a taste and/or smell in an animal or human. The flavouring agent may be natural, semi-synthetic or synthetic.

The flavouring agent may be a water soluble flavouring agent. The flavouring agent may be an oil soluble flavouring agent. The flavouring agent may be commercially available flavouring agent and may be selected depending on the use of the composition. Examples of flavouring agents include acids, amino acids, sugars, reducing sugars (e.g., ribose), vitamins, and minerals. Flavouring agents may also include microorganisms and fungi, such as recombinant microorganisms and yeast.

For example, the flavouring agent may be selected to mimic the taste and/or aroma of animal-derived meat when the composition is for use in a meat analogue such as meat flavouring agents.

The flavouring agent may be a sweet flavouring agent such as a sugar, or a compound that activates a T1R2/T1R3 receptor in vitro.

Colouring agents may be any suitable colouring that is safe for consumption by animals and humans. Examples of suitable colouring agents include titanium dioxide, calcium carbonate and calcium phosphate.

Examples of spices and seasonings include salts (such as sodium or calcium chloride), pepper and/or garlic. The amounts and types of spices and seasonings added may be determined by the type of meat analogue intended.

The Maillard reaction, also called non-enzymatic browning reaction, plays a role in food technology, on the one hand for the typical brown colour of roasted, baked or fried food products and on the other hand for the associated typical aroma. The Maillard reaction is not a single, specific chemical reaction, but a complex set of many reactions that lead to a large number of reaction products. A so-called reaction product is mainly responsible for the desired effects Melanoidins. These always occur in foods when compounds with free carbonyl groups, such as reducing sugars or lipid oxidation products or products from the degradation of polyphenols (o-quinones) and fermentative processes (methylglyoxal) with proteins, peptides and/or amino acids are heated together.

The single cell protein products (e.g. biomass, culture broth and/or protein thereof) may be able to undergo a colour change when heated, similar to the colour change seen when cooking meat. As such, meat analogues including the single cell protein products (e.g. biomass, culture broth and/or protein thereof) as described herein may be able to mimic the colour change seen when cooking animal meat and therefore be able to replicate the Maillard reaction.

The meat analogue may include a binder. Examples of binders include those described in EP0031622A1 and U.S. Pat. No. 4,125,630A.

The binder may comprise a cold-set binder, for example, a sodium alginate solution or a salt mixture solution.

The binder may comprise a heat-set binder, for example, a protein that denatures when heated. For example, the heat-set binder may comprise a soy protein, egg protein, potato protein, rubisco or a hydrocolloid, such as methyl cellulose. In examples where a heat-set binder is used, the meat analogue is heated to set the heat-set binder.

The binder may alternatively comprise an enzymatic binder as described herein, such as a transglutaminase enzyme. An enzymatic binder may form enzymatic crosslinking without the requirement to heat the meat analogue. As such, the meat analogue will not change colour due to heating when forming the meat analogue from or comprising the biomass or protein products derived therefrom as described herein.

The meat analogues described herein may also include a fat cell analogue as described herein that is separate from the binder. The meat analogues may comprise a fat analogue such as plant oils and oil gels formed from hydrophobic polymers, such as those described in Dreher, Johannes, et al. “Formation and characterisation of plant-based emulsified and crosslinked fat crystal networks to mimic animal fat tissue”. Journal of food science 85.2 (2020): 421-431. And Patel A. R., Nicholson R. A., Marangoni A. G. 2020. Applications of fat mimetics for the replacement of saturated and hydrogenated fat in food products. Current Opinion in Food Science, 33:61-68. In addition, fat analogues include those described in WO2014110539A1 which describes an adipose tissue analogue including one or more isolated plant proteins, and one or more plant or algal derived oils as well as those described in WO2008047012 which describes a fat analogue that is a mixture of semi-solid palm oil representing and at least one vegetable oil which is fluid at ambient temperature.

The meat analogue may be vegetarian. The meat analogue may be vegan. All of the components of the meat analogue described herein may be vegetarian or vegan. That is to say, the components are not made from or with the aid of products or components derived from animals that have died, have been slaughtered, or animals that die as a result of being eaten. The meat analogue described herein may be vegan. That is to say, the components are not sourced from or derived from an animal or animal product. Food products that are “vegan” are free of any animal products or animal by-products.

As single cell protein product (e.g. biomass or protein of the biomass and/or culture broth) described herein are for use in food products which are to be ingested by an animal, such as by humans, all the components are preferably food grade and/or food safe. Food grade refers to components that meet government food safety standards and have been approved for animal and/or human consumption. For example see the Food Chemicals Codex (FCC). In some examples, “food grade” refers to an agent or substance classified by the United States Food and Drug Administration (US FDA) as generally regarded as safe (GRAS). “Food grade” refers to an agent or substance that is a “feed material” or “food additive” as these terms are defined by the European Union. Food materials are defined, for example, by Regulations (EC) Nos 1831/2003, 767/2009, and 575/2011 of the European Parliament and of the Council. Food additives are defined, for example, by European Parliament and Council Directive 95/26 EC. As such, each of these agents or substances can be added directly to food or a feedstock, or used in food contact applications. Accordingly, their use is considered safe for animal and human consumption.

In some examples, the food product may include a single cell protein product with a relatively high concentration of cytochrome C. In such examples, any food product that includes the single cell protein product may have a red, pink, beige, off-white or reddish brown colour.

In some examples, a single cell protein product with a relatively high concentration of cytochrome C may be added to a food product as described herein in order to increase the level of cytochrome C in the food product.

In some examples, cytochrome C produced by the culturing methods described herein may be isolated or purified and added into a food product as described herein.

In some examples, the cytochrome C produced by methods described herein may be added to another single cell protein product, such as a single cell protein product as described herein. For example, a single cell protein product including a relatively high concentration of cytochrome C as described herein may be added to another single cell protein product produced by the methods described herein.

It will be understood that when single cell protein product including a relatively high concentration of cytochrome C as described herein is added to a food product or another single cell protein product, the concentration of cytochrome C will be less than for the single cell protein product including a relatively high concentration of cytochrome C as described herein due to dilution. As such a food product or another single cell protein product comprising single cell protein product including a relatively high concentration of cytochrome C as described herein may have a final concentration of cytochrome C of at least 10×10⁻³ mM or less than 10×10⁻³ mM depending on the starting concentration of cytochrome C and dilution factor.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

Aspects of the invention are demonstrated by the following non-limiting examples.

### EXAMPLES

### Abbreviations

DO Dissolved oxygen
FD Freeze drying
OD Optical density (at 600 nm)
OHC Oil holding capacity
SD Spray drying
UF Ultrafiltration
WHC Water holding capacity

### Example 1

### Materials and Methods

### Strains

Methylophilus methylotrophus DSM 5691 obtained from the German Collection of Microorganisms and Cell Cultures (DSMZ).

### Fermentation

### Culture Media

### Principle

Fed batch growth of Methylophilus methylotrophus (DSM 5691):
Inoculate shake flasks containing 200 mL each with Bourque medium 3.
Inoculate fermentor (8 L initial working volume) with Bourque medium 3 by choosing the culture with an OD600 nm between 2.0-2.5.

### Set-up

Fermentor equipped with pH sensor, oxygen sensor.
Connect base solution for pH control: pH 6.8.
Connect antifoam solution (Antifoam C, Sigma).
Connect the nutrient solution (AMS)·
DOT: 20% saturation using cascade mode.
Temperature: 37° C. . . .
Start the batch fermentation with a methanol concentration of 2.0 g/L.

### Medium Composition

1. Bourque medium 3 as preculture medium

Prepare the following medium and split it into two flasks with baffles.

### Bourque Medium 3:

| Component | Concentration (g/L) |
|---|---|
| KH2PO4 | 1.305 |
| Na2HPO4 or Na2HPO4•12H20 | 2.13 or 5.37 |
| (NH4)2SO4 | 1.500 |
| *MgSO4•7H2O | 0.450 |

*Prepare MgSO4•7H2O separately to avoid precipitation!

Then, add the four main components and autoclave it at 121° C. for 15 min. Cool down. Complete the volume until it reaches 200 mL, i.e., add a filter-sterilized solution of methanol to give a final concentration of 6 g/L. Combine this with the filter-sterilized concentrated trace element solution (10 mL/L), as follows:
195.48 mL of Bourque medium 3 (autoclavable)
2 mL of concentrated Bourque TE solution
1.52 mL of sterile methanol
1 mL 0.5% culture (20% glycerol cryovials)

### 2. Bourque Medium 3 as Fermentor Medium in the Bioreactor

For 8 L working volume in the reactor, weigh:

| Component | Mass (g) |
|---|---|
| KH2PO4 | 10.44 |
| Na2HPO4•12H2O | 42.96 |
| (NH4)2SO4 | 12 |
| *MgSO4•7H2O | 3.6 |

*Prepare MgSO4•7H2O separately to avoid precipitation!

Complete the medium until reach a mass of 7099.8 g. Autoclave at 121° C. for 20 min. Cool. Add a filter-sterilized solution of methanol to give a final concentration of 2 g/L. Combine this with a filter-sterilized concentrated trace element solution (10 mL/L) to complete the medium, as follows:
7099.8 g: Bourque medium 3 (autoclavable)

Add:
80 mL: Concentrated TE solution
20.2 mL: Sterile pure methanol
800 mL: Preinoculum
   3. Bourque trace elements solution (concentrated 100×):

The composition of the Bourque trace elements solution (concentrated 100×) is presented as follows. It is important to highlight the need of addition 1.5 mL of 25% HCl to the demi-water before start weighting all the components of the trace element solution to avoid precipitation.

| Component | Concentration (g/L) |
|---|---|
| FeSO4•7H2O | 2 |
| CaCl2•2H2O* dissolve seperately | 2 |
| MnSO4•H2O | 0.49 |
| ZnSO4•7H2O | 0.26 |
| Na2MoO4•2H2O | 0.08 |
| CuSO4•5H2O | 0.08 |
| CoCl2•6H2O | 0.08 |
| H3BO3 | 0.06 |

### 4.4 Methanol in the Fed-Batch Phase

Methanol purity 99.9% purity 1000 mL
   4.5 Acid Mineral Solution (AMS) composition used in the fed-batch phase

The composition of acid solution that was supplied in the fed-batch culture was determined as follows:

Acid Mineral Solution (AMS)

| Volume | | |
|---|---|---|
| 1000 mL | 500 mL | Component |
| 270 | 135 | g of H3PO4 |
| 100 | 50 | g of MgSO4•7H20* dissolve separately! |
| 2 | 1 | g of FeSO4•7 H20 |
| 2 | 1 | g of CaCl2•2 H20* dissolve separately! |
| 0.5 | 0.25 | g of MnSO4•H20 |
| 0.26 | 0.13 | g of ZnSO4•7 H20 |
| 0.08 | 0.04 | g of Na2MoO4•2H2O |
| 0.08 | 0.04 | g of CuCl2•2 H20 |
| 0.08 | 0.04 | g of CoCl2•6 H20 |
| 0.06 | 0.03 | g of H3BO3 |

### Pre-Culture

1 mL cryo-vial was inoculated in 2×200 mL Bourque medium supplemented with 6 g/L methanol in 2 L baffled shake-flasks with vented cap, and incubated at 300C in a rotary shaker operating at 150 rpm for ca. 19 hours.

### Unrestricted Growth: Batch Fermentation

### Restricted Growth: Fed-Batch Fermentation

### Harvest and Post-Processing

Once the fermentation was completed, the fermentation broth was harvested by removing it from the bioreactor through pumping or differential pressure into storage containers. The broth was subsequently centrifuged at 20 000 g for 10-15 min. Some samples were directly freeze dried, other samples were first washed with demi water and centrifuged again prior to freeze drying.

### Analyses

### Protein Content

Protein content was measured by the Dumas method. Total nitrogen was determined using a FlashEA 1112 NC analyzer. The protein content is then derived using a nitrogen conversion factor based on the amino acid profile. In this work, we used the same conversion factor as used for Pruteen¹ (Protein %=N %×4.88)

### Water Holding Capacity (WHC)

A known amount of dry sample was first hydrated with Milli-Q water and stirred overnight at 20 rpm. The sample was then centrifuged at 20 000 g and 25° C. for 10 min. The pellet was then weighed and dried overnight at 105° C. The WHO is calculated from the weight of the wet sample (pellet) relative to the dried sample.

1. Hydration with Water
2% w/v solution was made by dissolving 0.2 g dried single cell protein powder into 10 mL Milli-Q water in a 50 mL falcon tube, mixing with a vortex
Stirring overnight with a rotator at a speed of 20 rpm

### 2. Centrifugation

Centrifuge the falcon tube under 20,000G for 10 min at 25 degree.
Discard the supernatant
Weigh the wet pellet and empty aluminum pan
Transfer the wet pellet into the aluminum pan

### 3. Oven Drying.

Dry the sample at 105 C for overnight
Weigh the dried pellet with the pan.
Calculate the WHC, the WHC is calculated as the weight difference between the wet pellet and dry pellet divided by the weight of the dried pellet, with a unit of g water/g dry pellet

### Oil Holding Capacity (OHC)

A known amount of dry sample was dispersed in sunflower oil at 20 rpm. The sample was then centrifuged at 3050 g and 25° C. for 20 min. The pellet was then weighed. The OHC is calculated from the weight of the wet sample (pellet) relative to the initial dry sample.

### 1. Disperse the Sample in Oil

Make a 2% w/v dispersion, mix 0.2 g dried single cell protein powder and 10 mL sunflower oil in a 15 mL falcon tube
Stirring 30 min with a rotator at a speed of 20 rpm

### 2. Centrifugation

Centrifuge the falcon tube at 3050 g for 20 min at 25C
Discard the oil phase, place the tubes upside down on paper to drain the oil completely
Weigh the falcon tube with the wet pellet.

The OHC is calculated as the weight difference between the wet pellet and the weight of the original powder in dry matter divided by the weight of the original powder in dry matter.

### Rheological Behavior

Rheological behavior was determined through a temperature sweep in a closed cavity rheometer (RPA Flex, TA Instruments). A sample was brought to 40% dry matter concentration and placed in the rheometer, where first a heating cycle from 40 to 150° C. was performed, followed by a cooling down cycle from 150 to 50° C.

### Viscosity

1. Make 10% w/v dispersion, mix 0.4 g dried single cell protein powder with 4 mL water in a 15 mL falcon tube.
2. The viscosity was determined by an Anton Paar (TA instrument, The Netherlands) and a flow sweep was applied to the dispersions at 20° C. from 0.1 to 100 s−1 to measure the viscosity, and the result is obtained from the device with the unit of mPa/s.

### Results

Functional properties of microbial protein compared to plant protein

TABLE 1
Protein content and functional properties for plant proteins
and microbial biomass obtained from batch and fed-batch fermentation
as described in the Materials and Methods section.

| | Protein | | | |
|---|---|---|---|---|
| Sample¹ | content | WHC² | Solubility³ | OHC⁴ |
| PPI | 70% | 11.1 | 36.7 | 1.7 |
| PPC | 48% | 2.5 | 50.6 | 1.0 |
| SPI | 83% | 15.8 | 74.1 | 1.3 |
| SPC | 63% | 6.2 | 27.3 | 1.6 |
| Microbial | 45% | 6.2 | N.d.36+ | 2.5 |
| biomass | | | | |
| (batch) | | | | |
| Microbial | 69% | 6.6 | 20.4 | 2.4 |
| biomass (fed- | | | | |
| batch) | | | | |

¹PPI: pea protein isolate; PPC: pea protein concentrate; SPI: soy protein isolate; SPC: soy protein concentrate.
²WHC: water holding capacity, g/g dry pellet
³Expressed in percentage. N.d: not determined.
⁴OHC: oil holding capacity, g/g powder

Functional properties of microbial protein grown in batch and fed-batch mode

TABLE 2
Comparison of different cultivation modes

| Variable | Impact on functionality | Impact on application |
|---|---|---|
| Batch Fermentation | Functional properties similar to | Good for meat or dairy analogs |
| | plant based proteins | |
| Batch and fed-batch | OHC better than plant based | Good for oil and flavor binding |
| fermentation | proteins | in meat analogues. |
| Fed-batch fermentation | Improvement on rheology | Good for texturized products |
| | | like meat analogues |
| Fed-batch fermentation + post | Improvement on appearance | Good for meat analogues |
| processing | without texturization | |

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent, or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

### Example 2

Dutch startup Farmless raises €1.2M to turn renewable energy and CO₂ into carbon-negative food ingredients

Amsterdam, The Netherlands—May 10, 2023. Farmless, a Dutch startup, is using fermentation to produce carbon-negative, functional proteins with a complete amino acid profile. This technology, which will decouple food production from agricultural land, is backed by a €1.2 million pre-seed round led by Revent, Nucleus Capital, and Possible Ventures.
Fermentation platform is based on liquid feedstock made with CO₂, hydrogen and renewable energy.
Initial product is a functional protein that provides all essential amino acids and
Requires 500× less land than animal protein

Instead of using sugar, Farmless is pushing the fermentation frontier using a liquid feedstock which can be made with renewable energy. This makes Farmless' process dramatically more resource- and land efficient than animal farming, requiring 10-25× less land than plant protein and 250-500× less than animal protein.

The €1.2 million pre-seed round was co-led by Revent, Nucleus Capital, Possible Ventures with participation from HackCapital, Sustainable Food Ventures, VOYAGERS Climate-Tech Fund, TET Ventures and angels Jenny Saft through the Atomico Angel program, Ron Shigeta, Martin Weber, Rick Bernstein, Nadine Geiser, Joy Faucher, Michele Tarawneh, Alexander Hoffmann and Christian Stiebner.

“We're excited by the Farmless approach to fermentation. We're specifically interested in food technologies with high scalability and the potential to rapidly outperform on the cost per kg of protein. Across both of these dimensions Farmless shows great potential and it's a delight to work with this remarkable team,” says Lauren Lentz from Revent.

“We're proud to be backed by an amazing group of experienced climate tech investors who share our mission. With our fermentation platform we aim to dramatically outperform animal agriculture and reliably produce low-cost proteins at a planetary scale. We believe this technology has the potential to end factory farming, rewild our planet and draw down gigatons of carbon,” explains Adnan Oner, the founder and CEO of Farmless.

### a New Food Repertoire

Farmless' initial products are designed to replace animal proteins, as the majority of agricultural land is currently used to feed animals. The Farmless fermentation platform can potentially create a whole new food repertoire, producing proteins, carbohydrates, beneficial fats, vitamins, and minerals from the bottom up.

### About Farmless & The age of Electric Agriculture

Farmless' mission is to free food production from animals and agricultural land. The company's goal is to build a fermentation platform which can turn renewable electricity into food. Farmless was founded by Adnan Oner, a physicist and serial entrepreneur with a mission to create a future worth getting excited about.

### Example 3

### Interview Farmless

About you-What's your personal drive? Why is this company important to you? What makes you the perfect person to run this company?

A couple of years ago I wondered how to have the biggest possible impact with my next startup. So I asked myself: what are the biggest unsolved climate problems that few people are working on? I read lots of scientific literature to understand where all our carbon emissions were coming from and what technologies exist to reduce them. I found out that there are very few people thinking about making food production dramatically more land—and resource efficient. Efficient food production is crucial if we want to reverse centuries of agricultural sprawl and reforest the world. As a physicist I like to think about problems from first principles. So I got pretty excited when I found out there is a much better path to food than traditional agriculture: fermentation based on renewable electricity.

About the investors-Who are the lead investors, and what has it been like to work with them so far?

This round was co-led by Revent, Nucleus Capital, Possible Ventures with participation from HackCapital Sustainable Food Ventures, VOYAGERS Climate-Tech Fund, TET Ventures (and angels: Jenny Saft through the Atomico Angel program, Ron Shigeta, Martin Weber, Rick Bernstein, Nadine Geiser, Joy Faucher, Michele Tarawneh, Alexander Hoffmann and Christian Stiebner). We're proud to be backed by this large group of experienced climate tech investors who share our radical mission. They are as eager as us to find a reliable way to produce low-cost proteins at planetary scale, end factory farming and rewild our planet.

About the fundraise-What is the goal with this fundraise; what does this fundraise unlock and what are the next steps for the company?

With this round we were able to find microbes that taste and act like animal proteins, set up a lab and build a team of dedicated fermentation and food scientists. We're currently developing our initial product prototype which is an amino acid complete protein with high functionality. We're also pushing the boundaries of the performance of our fermentation process. Next steps would be moving to bigger fermentation vessels, building our supply chain, getting regulatory approval for our first product and bringing it to market with the right partners.

About the round-How much did you raise, at which valuation? What was the make-up of the round (equity/notes/debt, etc)

We raised a €1.2m pre-seed equity round at an undisclosed valuation.

About the short-term future-What are you personally most excited about for the next 18-24 months?

I'm thrilled to work with our multidisciplinary team on fine tuning our first products, which are designed to replace animal proteins. It is a super rewarding experience, working on the frontier of a new industry. I'm also excited about scaling up infrastructure and bringing our first product to market.

About the long-term future-What is the ultimate goal for your company? If everything goes to plan, how is the world different ten years from now, because your company exists?

We are building an interface between food and electricity, which means we are domesticating microbes selected for their food properties and their ability to grow on renewable energy-based feedstocks. The Farmless fermentation platform can potentially create a whole new food repertoire, producing proteins, carbohydrates, beneficial fats, vitamins, and minerals from the bottom up.

Here's the thing: instead of using sugar, Farmless is pushing the “fermentation frontier” using a liquid feedstock which can be made with renewable energy. This technology requires 10-25× less land than plant protein and 250-500× less than animal protein.

If everything goes to plan, we'll accelerate the advent of food fermentation in the next ten years. Our ultimate goal is to free food production from animals and agricultural land, so we can return vast amounts of land to rewild our planet, draw down carbon from the atmosphere and liberate animals from the food system. It's quite amazing that a single technology can achieve all these things at once.

What's hard-What (is/are) the biggest challenge(s) you are facing at the moment? What's the bottleneck for growth and progress?

Within Europe, regulatory uncertainty for sure. Within the EFSA the novel food procedure broadly has two phases, one is the food safety part, which is great. After receiving a favourable opinion from the EFSA, all member states get to vote, making it a highly political and unpredictable process.

At the moment public tastings of fermentation based products are not allowed under any conditions in The Netherlands where we are based, making it a more complicated process to get customer feedback before going through the regulatory process.

The bottleneck for future growth will be financing this first in kind infrastructure. Typical venture capital wants a high return on investment which does not match with infrastructural projects. These bridges need to be filled with project financing, ideally with governmental support to increase the transition to a more sustainable and affordable food system just like we did for renewables and electric vehicles.

People may wonder why you would want to take on agriculture at all?

People tend to have a very rosy view of agriculture—the happy little farm myth. But the reality-especially with animal agriculture—is that it is brutal towards animals and extremely wasteful in terms of resources and land use. Animal agriculture belongs in the same category as the fossil fuel industry. It causes so many bad things: biodiversity loss (>90% of tropical deforestation), CO₂ emissions, diseases, increase in antibiotic resistance, pesticide use, freshwater depletion, soil erosion, algae blooms—the list goes on and on. But it's hard to acknowledge this when we don't have any alternatives. I think that once we have alternatives to animal agriculture, people will start to see how bad most agriculture actually is.

### Example 4—Impact of culturing conditions on functionalities and color

### Materials & Methods

a) Strain

Methylophilus methylotrophus NCIMB10515 obtained from the National Collection of Industrial, Food and Marine Bacteria, UK (NCIMB)
b) Culturing conditions

Fed-batch cultivation in 1 L bioreactors with DO cascade at two set points, either 5% (Tanks 1 and 4) or 50% (Tanks 5 and 8), with increasing methanol (at 50% concentration) addition from 4 to 8 (Tanks 1 and 5) or to 20 (Tanks 4 and 8) g/kg/h.
c) Harvest and downstream processing

Centrifugation was done at 18000 g for 20 minutes. The pellet was collected and frozen for at least 24 hours prior to freeze drying.
d) Functional characterization

Color was qualitatively assessed on the pellet immediately after centrifugation and before freezing. WHC and OHC was determined as described in Example 1. Structuring (viscoelastic modulus) was tested using a closed cavity rheometer device, (RPA elite model from TA Instruments, USA). A 40% DM dried sample (biomass or culture broth) dispersion, weighing 5 grams, is sealed in a beaker with parafilm to minimize moisture loss and hydrated for at least 30 minutes before testing. The prepared sample is placed between two plastic foils within the CCR's closed cavity with disk geometry, under a constant 4 bar pressure to prevent evaporation. The device conducts temperature sweeps from 40° C. to 150° C. at a rate of 10° C./min, maintaining a 1% constant strain and 1 Hz frequency within the linear viscoelastic region. After reaching 150° C., the sample is cooled to 50° C. using dry air. The CCR records the storage modulus (G′) and loss modulus (G″), which are used to calculate the complex modulus (G*), indicative of the sample's viscoelastic behaviour and structural integrity post-cooling. Data analysis, performed by the rheometer's software, computes the complex shear modulus G* from G′ and G″, providing a quantitative measure of the sample dispersion's structural strength at the end of the cooling phase.

### Results

TABLE 3
Colour and functionality summary (WHC, OHC, and structuring
were assessed as described in Example 1).

| | | WHC | | |
|---|---|---|---|---|
| | Pellet | (g/g | OHC (g/g | Structuring |
| Dataset | colour | sample) | sample) | (Pa) |
| Tank 1 | Brown | 5.97 | 1.44 | 41.1 |
| Tank 4 | Brown/green | 4.70 | 1.73 | 15.27 |
| Tank 5 | Brown/pink | 3.29 | 1.66 | 18.29 |
| Tank 8 | Brown/pink | 5.57 | 2.11 | 2.59 |

These results show that both dissolved oxygen level and methanol feeding rate have an impact on the appearance and technical functionality of the single cell protein product. For example, a lower methanol feeding rate results in improved structuring properties (Tank 1 compared to Tank 4, and Tank 5 compared to Tank 8), as well as lower dissolved oxygen levels (Tank 1 compared to Tank 5, and Tank 4 compared to Tank 8).

EXAMPLE 5-Impact of harvesting conditions on functionality

Materials & Methods
a) Strain

Methylophilus methylotrophus DSM 5691 obtained from the German Collection of Microorganisms and Cell Cultures (DSMZ)
b) Culturing conditions

Fed-batch cultivation with similar media as described in Example 1. Cultivation was performed in a 15 L bioreactor with a DO cascade at 15% and linear methanol (at 100% concentration) addition of ca. 2 g/kg/h.
c) Harvest and downstream processing

The fermentation broth was split in several fractions to which varying combinations of DSP methods were applied.

Combination 1: Centrifugation and freeze drying. The broth was centrifuged at 20000 g for 15 minutes and the supernatant discarded. The pellet was scooped out, stored in boxes and frozen for at least 24 hours prior to freeze drying. Freeze drying was performed until a dry matter content of at least 95% was reached. Prior to functional characterization, the material was ground.

Combination 2: Centrifugation and spray drying. The broth was harvested in a similar manner as Combination 1, but instead of freeze drying, the pellet was thawed and spray dried in a mini Buchi spray drying. The obtained powder was used without further treatment for functional characterization.

Combination 3: Flocculation by acidification, centrifugation and freeze drying. The broth was brought to pH 4.5 by addition of phosphoric acid, and then centrifuged at 6500 g for 25 minutes. The pellet was further processed in the same way as in Combination 1.

Combination 4: Flocculation by acidification, centrifugation, neutralization and freeze drying. The broth was acidified and centrifuged in the same way as in Combination 3. The pellet was then neutralized to pH 6.5 by addition of ammonium hydroxide. The pellet was further processed in the same manner as in Combination 1.

Combination 5: Flocculation by flocculant, centrifugation and freeze drying. The broth was flocculated by addition of CaCl₂), and then centrifuged and freeze dried in the same way as for Combination 3.
d. Functional characterization

All materials were assessed on water holding capacity (WHC), oil holding capacity (OHC) and solubility in a similar way as described in Example 1.

Structuring (viscoelastic modulus) was tested using a closed cavity rheometer device, (RPA elite model from TA Instruments, USA). A 40% DM dried sample (biomass or culture broth) dispersion, weighing 5 grams, is sealed in a beaker with parafilm to minimize moisture loss and hydrated for at least 30 minutes before testing. The prepared sample is placed between two plastic foils within the CCR's closed cavity with disk geometry, under a constant 4 bar pressure to prevent evaporation. The device conducts temperature sweeps from 40° C. to 150° C. at a rate of 10° C./min, maintaining a 1% constant strain and 1 Hz frequency within the linear viscoelastic region. After reaching 150° C., the sample is cooled to 50° C. using dry air. The CCR records the storage modulus (G′) and loss modulus (G″), which are used to calculate the complex modulus (G*), indicative of the sample's viscoelastic behaviour and structural integrity post-cooling. Data analysis, performed by the rheometer's software, computes the complex shear modulus G* from G′ and G″, providing a quantitative measure of the sample dispersion's structural strength at the end of the cooling phase.

### Results

TABLE 4
Results overview and comparison to plant proteins

| | Protein | WHC (g/g | | OHC (g/g | Structuring |
|---|---|---|---|---|---|
| Sample | content | sample) | Solubility | sample) | (Pa) |
| PPI | 70% | 11.1 | 36.7 | 1.7 | 52.4 |
| PPC | 48% | 2.5 | 50.6 | 1.0 | 76.7 |
| SPI | 83% | 15.8 | 74.1 | 1.3 | 90.1 |
| SPC | 63% | 6.2 | 27.3 | 1.6 | 147.8 |
| Comb 1 | 88% | 8.5 | 50.9 | 2.2 | N.A |
| Comb 2 | 85% | 4.0 | 15.1 | N.A. | 78.9 |
| Comb 3 | 82% | 2.8 | 25.2 | 1.6 | 32.0 |
| Comb 4 | 81% | 5.3 | 20.2 | 2.0 | 30.6 |
| Comb 5 | 82% | 6.0 | 32.7 | 1.9 | 28.9 |

These results show that specific functional properties can be tuned through DSP combinations, depending on the target product formulation. For example, Combination 2 (centrifugation+spray drying) improves the structuring properties of the material, to a similar level to that of PPC, making it suited for meat alternatives.

### Example 6—Quantification of Cytochrome c in Culture Broth and in Single Cell Protein Product

### Materials & Methods

a) Quantification in single cell protein product, sample preparation.

1.5 grams of single cell protein product obtained by centrifugation and freeze drying in Example 5 (combination 1) was mixed with 36 g water and rehydrated for at least 12 hours. The suspension was then lysed through 5 cycles of 40 s sonication followed by 20 s rest, keeping the sample in an ice bath. 10 mL of the lysed suspension was then heated for 3 min in a water bath kept at 85° C., after which it was centrifuged at 18000 g for 15 min. The supernatant was kept for spectrophotometric analysis as described below.
b) Quantification in culture broth, cultivation conditions.

All experiments were performed with a strain, media and cultivation procedures as described in Example 4 with DO cascade at one of three set points, either 5% (Tank 1), 30% (Tanks 2 and 4) or 50% (Tank 3), with increasing methanol (at 50% concentration) addition from 3 to 12 (Tanks 1, 2 and 3) or from 4 to16 (Tank 4) g/kg/h. Cultivation proceeded for 48 hours.

Two cultivations were performed following a similar cultivation protocol as described by Jia et al 2024, as an example of prior art cultivation conditions. In short, a fed-batch protocol was performed and 1 L media, with a programmed methanol feed to maintain a residual methanol concentration of around 5 g/L, and a DO cascade set at 30%. In a similar fashion as described in the paper, when the DO dropped below 5% a constant feed of 2.5 g/kg/h was applied. Cultivation proceeded for 48 hours.

c) Quantification in culture broth, sample preparation.

Upon completion of the cultivation, 200 ml of culture broth were lysed through 5 cycles of 40 s sonication followed by 20 s rest, keeping the sample in an ice bath. 10 mL of the lysed suspension was then heated for 3 min in a water bath kept at 85° C., after which it was centrifuged at 18000 g for 15 min. The supernatant was kept for spectrophotometric analysis as described below.

The state-of-the-art cultivations resulted in significantly less biomass and hence, sample preparation was adapted to reach measurable levels of cytochrome c. The culture broth was centrifuged at 18000 g for 15 min, and the pellet (containing the produced biomass) was resuspended in water to reach a dry matter content of ca. 1.5%. This suspension was then lysed and further processed as the samples above.
d) Spectrophotometric analysis

Supernatant cytochrome c concentration in mM was obtained by following the procedure by Appaix et al, Biochimica et Biophysica Acta 1457 (2000). In short, 1.5 mL of supernatant was poured into a cuvette. The sample was then fully oxidized by adding 50 microliter of 0.03% H2O2. A spectrum scan was made in the wavelength range from 380 to 800 nm and the absorbance at the Soret peak (typically at around 414 nm) was recorded, and converted to mM through the extinction coefficient of oxidized cytochrome c. This is the concentration in the supernatant. For concentration in pellet or single cell protein product, cytochrome c concentrations in supernatant were then normalized to the dry mass of the original sample.

### Results

TABLE 5
Results in supernatant of culture broth

| | Cytochrome c |
|---|---|
| | concentration in supernatant |
| Sample | (×10\hat{ } − 3 mM) |
| Tank 1 | 17.21 |
| Tank 2 | 20.54 |
| Tank 3 | 27.88 |
| Tank 4 | 14.51 |
| Prior art method | 1.76 |

TABLE 6
Results in pellet (harvested biomass) and single cell
protein product (harvested and processed biomass)

| | Cytochrome c |
|---|---|
| | concentration (pmol/mg |
| Sample | biomass) |
| Tank 1 | 331.0 |
| Tank 2 | 427.9 |
| Tank 3 | 412.4 |
| Tank 4 | 245.9 |
| Comb 1 (Example 5) | 342.8 |

The results show that cultivation conditions as described herein (Tank 1-Tank 4) result in cytochrome c concentrations in pellet of at least 200 μmol/mg dry matter, and cytochrome c concentrations in supernatant of at least 10×10⁻³ mM.

The cytochrome c concentrations obtained by this method is still measurable in the single cell protein product (combination 1, Example 5).

Culture broth obtained by the methods described herein resulted in a total cytochrome c concentration in supernatant 8 to 16 times larger than the prior art method, at the same cultivation time.

1. A method of producing a single cell protein product for incorporation into a food product for human consumption, the single cell protein product comprising single cell protein and a concentration of Cytochrome C of at least 10×10⁻³-mM, the method comprising;
culturing the methylotrophic microorganism in a culture medium comprising methanol at a dissolved oxygen concentration from about 15% to 50%;
wherein the culturing conditions are configured to induce the methylotrophic microorganism to produce protein and Cytochrome C within a biomass and/or the culture medium to produce a culture broth; and/or
to provide the single cell protein product with properties configured for use in the food product:
wherein the culture broth comprises the concentration of Cytochrome C of at least 10×10⁻³-mM and/or wherein the biomass comprises a concentration of Cytochrome C of at least 200 pmol/mg of dry material of biomass.

2. The method of claim 1, wherein the method further comprises harvesting the single cell protein product, the harvesting comprises at least partially disrupting the cells of the methylotrophic microorganism in order to release the cellular components thereof; optionally wherein at least partially disrupting the cells comprises:
a. acidifying the cultured biomass and/or culture broth prior to separating the cultured biomass from the culture broth to provide an acidified culture broth comprising at least a portion of the Cytochrome C; optionally
wherein acidification is done in the same vessel as the culturing or in a separate vessel as the culturing; further optionally
wherein acidification results in a pH in the range 3 to 6;
b. homogenising the cultured biomass and/or culture medium (culture broth); and/or
c. heating the cultured biomass and/or culture medium (culture broth); and/or
d. Enzymatic treatment of the cultured biomass and/or culture medium (culture broth)

3. The method of claim 1 or 2, wherein the harvesting comprises centrifuging the culture broth or the at least partially disrupted cells at a force of more than 2000 g to provide a supernatant comprising the concentration of Cytochrome C of at least 10×10⁻³-mM and a pellet comprising the biomass;
wherein the pellet comprises the concentration of Cytochrome C of at least 200 pmol/mg of dry material of biomass.

4. The method of any preceding claim, wherein the method further comprises processing:
a. the protein of the biomass;
b. the protein of the culture broth;
c. the biomass; and/or
d. the culture broth; or
the harvesting further comprises processing:
a. the protein of the biomass;
b. the protein of the culture broth;
c. the biomass;
d. the culture broth;
e. the acidified culture broth; and/or
f. the supernatant.

5. The method of claim 4, wherein processing comprises filtration of the supernatant to provide a concentrated retentate.

6. The method of claim 5, wherein the method further comprises freezing the concentrated retentate.

7. The method of claim 5 or 6, wherein the method further comprises drying the concentrated retentate; optionally wherein drying comprises freeze drying and/or spray drying and or other drying methods.

8. A method of producing a single cell protein product for incorporation into a food product for human consumption, the single cell protein product comprising single cell protein, the method comprising;
culturing one or more methylotrophic microorganism in a culture medium comprising methanol under culturing conditions for fermentation of the methanol;
wherein the culturing conditions are configured to induce the methylotrophic microorganism to produce protein within a biomass and/or the culture medium to produce a culture broth; and/or
to provide the single cell protein product with properties configured for use in the food product.

9. The method of claim 8, wherein the single cell protein product comprises the biomass, the culture broth and/or the protein of the biomass and/or culture broth.

10. The method of claim 8 or 9, wherein the culturing conditions comprise exposing the methylotrophic microorganism to a dissolved oxygen concentration of about 5% to about 50%

11. The method of claim any preceding, wherein the culturing conditions comprise exposing the methylotrophic microorganism to one or more conditions during culturing selected from:
i) pH;
ii) temperature;
iii) methanol concentration;
iv) nitrogen concentration;
v) phosphorus concentration; and/or
vi) magnesium concentration optionally
wherein the one or more conditions comprises or further comprises one or more of:
aeration rate;
stirring rate;
nitrogen feed rate;
methanol feed rate; and/or
nutrient feed rate; further optionally
wherein:
the methanol concentration comprises 0-10 g/L;
the dissolved oxygen concentration comprises 15 to 45%;
the pH comprises a pH from about 5 to 7;
the nitrogen concentration comprises 0-1.2 g/L;
the temperature comprises from 20 to 70° C.;
the phosphorus concentration comprises from 0 to 2.0 g/l; and/or
the magnesium concentration comprises from 0 to 80 mg/l.

12. The method of any preceding claim, wherein the culturing conditions comprise stress conditions; optionally wherein the stress conditions comprise nitrogen limitation and/or oxygen limitation.

13. The method of any preceding claim, wherein the methanol comprises sustainable methanol, optionally one or more of e-methanol, bio-methanol, blue methanol and/or grey methanol.

14. The method of any preceding claim, wherein culturing comprises restricted fermentation and/or unrestricted fermentation; optionally wherein the culturing comprises:
a. a batch fermentation process;
b. a fed-batch fermentation process;
c. a continuous fermentation process;
d. a feed-and-draw fermentation process;
e. a perfusion fermentation process;
f. a sequential batch process; and/or
g. a cell recycle fermentation process.

15. The method of any preceding claim, wherein the methylotrophic microorganism comprises one or more of at least one yeast, at least one bacteria and/or at least one archaea;
optionally wherein the methylotrophic yeast is one or more selected from the group consisting of Acidomonas, Candida, Hansenula, Komagataella, Ogataea, Pichia, Rhodotorula, and/or Torulopsis;
optionally wherein the methylotrophic bacterium is one or more selected from the group consisting of Acidomonas, Albibacter, Amycolatopsis, Ancylobacter, Arthrobacter, Bacillus, Chenggangzhangella, Clostridium, Hydrogenovibrio, Hyphomicrobium, Labrys, Leisinglera, Methanococcoides, Methanothermobacter, Methylacidiphilum, Methylibium, Methylobacillus, Methylobacterium, Methylorubrum, Methylocaldum, Methylocapsa, Methyloceanibacter, Methylocella, Methylococcus, Methylocystis, Methyloferula, Methylogaea, Methyloglobulus, Methylohalobius, Methylohalomonas, Methylomicrobium, Methylomonas, Methylonatrum, Methylophaga, Methylophilus, Methylopila, Methylorosula, Methylorubrum, Methylosarcina, Methylosinus, Methylosoma, Methylosphaera, Methylotenera, Methylothermus, Methyloversatilis, Methylovirgula, Methylovorus, Microvirga, Nocardia, Novimethylophilus, Oharaeibacter, Paracoccus, Pontibaca, Pseudomonas, Pyrinomonas, Rhodoblastus, Rhodococcus, Rubrivivax, Shinella, and/or Sporomusa;
optionally wherein the methylotrophic archaea is one or more selected from Methanohalophilus, Methanolacinia, Methanolinea, Methanolobus, Methanomethylovorans, Methanosarcina, Methanobrevibacter, Methanothermococcus, Methanococcus, Methanotorris, Methanothermobacter Methanofervidicoccus, Methanosphaera, and/or Methanimicrococcus;
optionally wherein the methylotrophic microorganism is a strain of Methylophilus methylotrophus.

16. The method of any preceding claim, wherein the method further comprises harvesting the cultured protein of the biomass and/or culture broth or harvesting the cultured biomass.

17. The method of claim 16, wherein harvesting comprises separating the protein from the cultured biomass and/or culture broth or separating the biomass from the culture broth, optionally wherein separating comprises one or more of flocculation, flotation, decanting, filtration and/or centrifugation.

18. The method of claim 16 or 17, wherein the harvesting comprises at least partially disrupting the cells of the methylotrophic microorganism prior to separating in order to release the cellular components thereof; optionally wherein at least partially disrupting the cells comprises:
a. acidifying the cultured biomass and/or culture broth prior to separating the cultured biomass from the culture broth to provide an acidified culture broth;
b. homogenising the cultured biomass and/or culture medium (culture broth); and/or
c. heating the cultured biomass and/or culture medium (culture broth) and/or
d. Enzymatic treatment of the cultured biomass and/or culture medium (culture broth)

19. The method of any preceding claim, wherein the harvesting further comprises washing prior to separation and/or washing the separated biomass.

20. The method of any of claims 16 to 19, wherein the harvesting further comprises drying the separated protein of the biomass and/or culture broth or drying the harvested biomass.

21. The method of any of preceding claim, wherein the methylotrophic microorganism is cultured using an unrestricted fermentation process and the single cell protein product has a viscosity of less than 200 mPa·s measured at 1S⁻¹; or
wherein the methylotrophic microorganism is cultured using a restricted fermentation process and the single cell protein product has a viscosity of more than 300 mPa·s measured at 1S⁻¹.

22. The method of any preceding claim wherein the method further comprises processing
a. the protein of the biomass;
b. the protein of the culture broth;
c. the biomass; and/or
d. the culture broth; or
the harvesting further comprises processing:
a. the protein of the biomass;
b. the protein of the culture broth;
c. the biomass; and/or;
d. the culture broth;
using processing methods configured to provide properties to the single cell protein product configured for use in the food product; optionally wherein the processing comprises one or more of:
(a) centrifugation;
(b) homogenisation;
(c) acidification;
(d) drying;
(e) enzymatic treatment;
(f) cooling;
(g) heating;
(h) filtration;
(i) rehydrating;
(j) rotary evaporation;
(k) acid gelation;
(l) curdling;
(m) texturizing; and/or
(n) freezing.

23. The method of claim 22, wherein processing comprises;
a. centrifugation at force less than 10,000 g, optionally wherein the protein of the biomass and/or culture broth or the biomass and/or culture broth (single cell protein product) has a viscosity of less than 200 mPa·s measured at 1S⁻¹ prior to centrifugation; optionally wherein centrifugation is at a force of less than 6500 g;
b. homogenisation and centrifugation at a force less than 10,000 g, optionally wherein the protein of the biomass and/or culture broth or the biomass and/or culture broth (single cell protein product) has a viscosity of more than 300 mPa·s measured at 1S⁻¹ prior to homogenisation; optionally wherein centrifugation is at a force of less than 6500 g; or
c. centrifugation at a force more than 10,000 g, optionally wherein the protein of the biomass and/or culture broth or the biomass and/or culture broth (single cell protein product) has a viscosity of more than 300 mPa·s measured at 1S⁻¹ prior to centrifugation; optionally wherein centrifugation is at a force of 18000 g.

24. The method of any of claims 17 to 19 and 21 to 23, wherein the method further comprises drying the centrifuged protein of the biomass and/or culture broth or the biomass and/or culture broth.

25. The method of any of claims 20 to 24, wherein the method further comprises rehydrating the dried protein of the biomass and/or culture broth or the dried biomass and/or dried culture broth.

26. The method of any of claims 17 to 25, wherein supernatant from any of the centrifugations is acidified.

27. The method of claim 26, wherein the acidified supernatant is further centrifuged at a force less than 10,000 g.

28. The method of any preceding claim, wherein the properties are selected from one or more of: rheological properties, protein concentration, aesthetic properties, organoleptic properties, structural properties and/or physical properties;
optionally wherein the food product is:
I) dairy analogue and the single cell protein product comprises a viscosity of less than 200 mPa·s measured at 1S⁻¹, a cream like texture and/or a white or off white colour;
II) a meat analogue and the single cell protein product comprises a viscosity of more than 300 mPa·s measured at 1S⁻¹, an oil holding capacity (OHC) of more than 1.5 grams of oil per gram of single cell protein product, organoleptic properties mimetic of meat and/or appearance mimetic of meat.

29. A single cell protein product produced by a method according to any preceding claim.

30. The single cell protein product of claim 29, wherein the single cell protein product comprises a protein content determined by a Dumas method with a Nitrogen Conversion Factor of 4.88 of at least 45%.

31. The single cell protein product of any of claims 29 to 30, wherein:
a. the microorganism has been cultured using an unrestricted fermentation process and wherein the single cell protein product comprises:
i) viscoelastic properties;
ii) a protein content of about 45%;
iii) a water holding capacity of at least 2.5 gram water per gram single cell protein product;
iv) an oil holding capacity of at least 1.0 gram oil per gram of single cell protein product;
v) a creamy texture;
vi) a white colour;
vii) a viscosity of less than 200 mPa·s measured at 1S⁻¹ and/or
viii) solubility of at least 20%.
b. the microorganism has been cultured using a restricted fermentation process and wherein the single cell protein product comprises:
i) viscoelastic properties;
ii) a protein content of about 60%;
iii) a water holding capacity of about 6 gram water per gram of single cell protein product;
iv) an oil holding capacity of about 1.5 gram water per gram of single cell protein product;
v) a colour and/or appearance mimetic of animal meat;
vi) a viscosity of more than 300 mPa·s measured at 1S⁻¹; and/or
vii) solubility of about 20%.

32. A single cell protein product for incorporation into a food product for human consumption, the single cell protein product comprising a concentration of Cytochrome C of at least 10×10⁻³ mM; and wherein the single cell protein product comprises or is obtained from a culture broth produced by culturing a methylotrophic microorganism.

33. The single cell protein product of claim 32, wherein the single cell protein product is produced by a method according to any one of claims 1 to 7 and 11 to 31.

34. The single cell protein product of claim 32 or 33, wherein the Cytochrome C is a product of culturing the methylotrophic microorganism.

35. The single cell protein product of any of claims 32 to 34, wherein the Cytochrome C has not been added from an exogenous source.

36. The single cell protein product of any one of claims 32 to 35, wherein the methylotrophic microorganism has been cultured using a fed-batch or continuous cultured method and a dissolved oxygen concentration from about 15% to 50%.

37. The single cell protein product of any of claims 32 to 36, wherein the single cell protein product has been harvested and/or processed according to any one of claims 2 to 7 and 22 to 28.

38. The single cell protein product of any one of claims 32 to 37, wherein the culture broth comprises cultured microorganism cells and culture media or a product derived from the culture broth and wherein the culture broth or product derived therefrom comprises the concentration of Cytochrome C of at least 10×10⁻³ mM.

39. The single cell protein product of claim 38, wherein the culture broth has been centrifuged and wherein the supernatant comprises at least a portion of the Cytochrome C.

40. A methylotrophic microorganism biomass or product derived therefrom comprising a concentration of Cytochrome C of at least 200 pmol/mg of dry material of biomass or wherein the microorganism produces or has produced a concentration of Cytochrome C of at least 200 pmol/mg of dry material of biomass.

41. The methylotrophic microorganism biomass according to claim 40, wherein the microorganism has been cultured according to any one of claims 1 to 7 and 11 to 31.

42. A food product for human consumption comprising a single cell protein product according to any one of claims 29 to 39 or the methylotrophic microorganism biomass according to claim 40 or 41.

43. The food product of claim 42, wherein the food product comprises:
a. a dairy analogue;
b. an egg analogue;
c. a beverage;
d. a soup;
e. a sauce;
f. a dairy product;
g. a seafood analogue; and/or
h. a meat analogue.

44. The food product of claim 42 or 43, wherein the single cell protein product is according to claim 31 (a) and wherein the food product comprises
a. a dairy analogue;
b. an egg analogue;
c. a beverage;
d. a soup;
e. a sauce;
f. a seafood analogue; and/or
g. a dairy product; or
wherein the single cell protein product is according to claim 31 (b) and
wherein the food product comprises a meat analogue.

45. The single cell protein product of any one of claims 32 to 39 or the food product of claim 42 or 44, wherein the single cell protein product or food product comprises a pink, red, beige, off-white or reddish-brown colour.

46. Use of sustainable methanol in a method of producing a composition comprising protein, a biomass or a single cell protein product cultured from a methylotrophic microorganism; optionally wherein the methanol is e-methanol; further optionally wherein the method comprises a method according to any one of claims 1 to 28.
